SEQUENCE LISTING (22 sequences)

SEQ ID NO: 1 - protein, 10 aa, synthetic construct
GFTFSQYAMA

SEQ ID NO: 2 - protein, 17 aa, synthetic construct
GISPYGSNTRYADSVKG

SEQ ID NO: 3 - protein, 11 aa, synthetic construct
NGGYSYTWFDL

SEQ ID NO: 4 - protein, 11 aa, synthetic construct
RASQSISSDLA

SEQ ID NO: 5 - protein, 7 aa, synthetic construct
WASKLQS

SEQ ID NO: 6 - protein, 9 aa, synthetic construct
MQMYNTPPT

SEQ ID NO: 7 - protein, 120 aa, synthetic construct
EVQLVESGGGLVQPGGSLRLSCAASGFTFSQYAMAWVRQAPGKGLEWVSGISPYGSNTRYADSVKGRFTISRDNSKNTLYLQMNSLRAEDTAVYYCARNGGYSYTWFDLWGQGTLVTVSS

SEQ ID NO: 8 - protein, 108 aa, synthetic construct
DIQMTQSPSSLSASVGDRVTITCRASQSISSDLAWYQQKPGKAPKLLIYWASKLQSGVPSRFSGSGSGTDFTLTISSLQPEDFATYYCMQMYNTPPTFGQGTKVEIKR

SEQ ID NO: 9 - protein, 705 aa, synthetic construct
EVQLVESGGGLVQPGGSLRLSCAASGFTFSQYAMAWVRQAPGKGLEWVSGISPYGSNTRYADSVKGRFTISRDNSKNTLYLQMNSLRAEDTAVYYCARNGGYSYTWFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGAEVKKPGASVKVSCKASGYTFTRYTMHWVRQAPGQGLEWIGYINPSRGYTNYNQKFKDKATLTTDKSISTAYMELSRLRSDDTAVYYCARYYDDHYTLDYWGQGTLVTVSSGGGGSGGGGSGGGGSEIVLTQSPATLSLSPGERATLSCSASSSVSYMNWYQQKPGQAPRRWIYDTSKLASGIPARFSGSGSGTSYTLTISSLEPEDFAVYYCQQWSSNPFTFGQGTKVEIKR

SEQ ID NO: 10 - protein, 449 aa, synthetic construct
EVQLVESGGGLVQPGGSLRLSCAASGFTFSQYAMAWVRQAPGKGLEWVSGISPYGSNTRYADSVKGRFTISRDNSKNTLYLQMNSLRAEDTAVYYCARNGGYSYTWFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 11 - protein, 214 aa, synthetic construct
DIQMTQSPSSLSASVGDRVTITCRASQSISSDLAWYQQKPGKAPKLLIYWASKLQSGVPSRFSGSGSGTDFTLTISSLQPEDFATYYCMQMYNTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 16 aa, synthetic construct
GGGGGSGGGGSGGGGS

SEQ ID NO: 13 - protein, 10 aa, synthetic construct
GYTFTRYTMH

SEQ ID NO: 14 - protein, 13 aa, synthetic construct
YINPSRGYTNYNQ

SEQ ID NO: 15 - protein, 10 aa, synthetic construct
YYDDHYTLDY

SEQ ID NO: 16 - protein, 8 aa, synthetic construct
SSSVSYMN

SEQ ID NO: 17 - protein, 7 aa, synthetic construct
DTSKLAS

SEQ ID NO: 18 - protein, 9 aa, synthetic construct
QQWSSNPFT

SEQ ID NO: 19 - protein, 241 aa, synthetic construct
QVQLVQSGAEVKKPGASVKVSCKASGYTFTRYTMHWVRQAPGQGLEWIGYINPSRGYTNYNQKFKDKATLTTDKSISTAYMELSRLRSDDTAVYYCARYYDDHYTLDYWGQGTLVTVSSGGGGSGGGGSGGGGSEIVLTQSPATLSLSPGERATLSCSASSSVSYMNWYQQKPGQAPRRWIYDTSKLASGIPARFSGSGSGTSYTLTISSLEPEDFAVYYCQQWSSNPFTFGQGTKVEIKR

SEQ ID NO: 20 - protein, 500 aa, synthetic construct
EVQLVESGGGLVQPGGSLRLSCAASGFTFSQYAMAWVRQAPGKGLEWVSGISPYGSNTRYADSVKGRFTISRDNSKNTLYLQMNSLRAEDTAVYYCARNGGYSYTWFDLWGQGTLVTVSSGGGGGSGGGGSGGGGSDIQMTQSPSSLSASVGDRVTITCRASQSISSDLAWYQQKPGKAPKLLIYWASKLQSGVPSRFSGSGSGTDFTLTISSLQPEDFATYYCMQMYNTPPTFGQGTKVEIKRGGGGSGGGGSGGGGSQVQLVQSGAEVKKPGASVKVSCKASGYTFTRYTMHWVRQAPGQGLEWIGYINPSRGYTNYNQKFKDKATLTTDKSISTAYMELSRLRSDDTAVYYCARYYDDHYTLDYWGQGTLVTVSSGGGGSGGGGSGGGGSEIVLTQSPATLSLSPGERATLSCSASSSVSYMNWYQQKPGQAPRRWIYDTSKLASGIPARFSGSGSGTSYTLTISSLEPEDFAVYYCQQWSSNPFTFGQGTKVEIKR

SEQ ID NO: 21 - protein, 15 aa, synthetic construct
GGGGSGGGGSGGGGS

SEQ ID NO: 22 - protein, 499 aa, synthetic construct
EVQLVESGGGLVQPGGSLRLSCAASGFTFSQYAMAWVRQAPGKGLEWVSGISPYGSNTRYADSVKGRFTISRDNSKNTLYLQMNSLRAEDTAVYYCARNGGYSYTWFDLWGQGTLVTVSSGGGGSGGGGSGGGGSDIQMTQSPSSLSASVGDRVTITCRASQSISSDLAWYQQKPGKAPKLLIYWASKLQSGVPSRFSGSGSGTDFTLTISSLQPEDFATYYCMQMYNTPPTFGQGTKVEIKRGGGGSGGGGSGGGGSQVQLVQSGAEVKKPGASVKVSCKASGYTFTRYTMHWVRQAPGQGLEWIGYINPSRGYTNYNQKFKDKATLTTDKSISTAYMELSRLRSDDTAVYYCARYYDDHYTLDYWGQGTLVTVSSGGGGSGGGGSGGGGSEIVLTQSPATLSLSPGERATLSCSASSSVSYMNWYQQKPGQAPRRWIYDTSKLASGIPARFSGSGSGTSYTLTISSLEPEDFAVYYCQQWSSNPFTFGQGTKVEIKR

(19) United States
(12) Patent Application Publication
(10) Pub. No.: US 2025/0019426 A1
(43) Pub. Date: Jan. 16, 2025

(54) ANTI-GRP78 ANTIBODIES AND THEIR USES

(71) Applicant: UCT BIOSCIENCE CO., LTD., TAIPEI CITY (TW)

(72) Inventors: CHIA-CHENG WU, TAIPEI CITY (TW); YA-WEI TSAI, TAIPEI CITY (TW); TZU-YIN LIN, TAIPEI CITY (TW); CHIA-HSIANG LO, TAIPEI CITY (TW)

(73) Assignee: UCT BIOSCIENCE CO., LTD., TAIPEI CITY (TW)

(21) Appl. No.: 18/762,879

(22) Filed: Jul. 3, 2024

Related U.S. Application Data
(60) Provisional application No. 63/511,711, filed on Jul. 3, 2023.

(51) Int. Cl. C07K 16/18; C07K 16/28

(52) U.S. Cl. CPC C07K 16/18; C07K 16/2809; C07K 2317/24; C07K 2317/622; C07K 2317/92

(57) ABSTRACT

The present disclosure relates to an anti-glucose-regulated protein 78 (GRP78) antibody or an antigen-binding fragment thereof. The present disclosure also relates to a method for treating and/or preventing a disease and/or disorder caused by or related to GRP78 activity or signaling, and a method or kit for detecting GRP78 or a cancer in a sample.

### PRIORITY INFORMATION

The subject application claims priority to and benefit of U.S. Provisional Patent Application No. 63/511,711, filed Jul. 3, 2023, the content of which is incorporated herein by reference in its entirety.

### REFERENCE TO ELECTRONIC SEQUENCE LISTING

The instant application contains a Sequence Listing which is submitted electronically in .xml format and is hereby incorporated by reference in its entirety. Said .XML copy, created on Jul. 3, 2024, is named “US14709-SEQ.xml” and is 22,000 bytes in size.

### FIELD OF THE INVENTION

The present disclosure relates to novel antibodies, particularly to antibodies which is specific for glucose-regulated protein 78 (GRP78). The present disclosure also relates to uses of such antibodies for suppressing tumor growth and metastasis.

### BACKGROUND OF THE INVENTION

Cancer is a leading cause of death and an important barrier to increasing life expectancy worldwide. Cancer targeted drug delivery and therapeutic approaches aimed at tumors hold promise in diminishing or halting tumor progression in living organisms, enabling them to enjoy extended and healthier lifespans. Nevertheless, numerous anti-tumor medications also exhibit toxicity towards non-tumor cells, leading to challenging and undesirable side effects. Consequently, there exists a requirement within the field for a method to selectively deliver anti-tumor agents exclusively to tumor cells, with the objective of minimizing tumor cell proliferation.

Glucose-regulated protein 78 (GRP78) is primarily a chaperone protein located within the endoplasmic reticulum (ER), assisting in the proper folding of proteins by aiding misfolded protein targeting and facilitating protein assembly. However, under cellular stress, GRP78 is transported to the cell surface (csGRP78), where it interacts with various ligands and initiates diverse intracellular pathways. Consequently, the expression of csGRP78 is associated with cancer and contributes to the maintenance and progression of the disease. Notably, the external exposure of csGRP78 prompts the production of autoantibodies, as observed in patients with prostate or ovarian cancer, indicating that the ability to target csGRP78 has an impact on tumor development. Given that csGRP78 is present on the surface of cancer cells but not normal cells in vivo, it represents a promising target for therapeutic antibody approaches.

However, the production of antibodies specific to GRP78 can be challenging. GRP78 is expressed on the cell surface of embryonic stem cells and homozygote knockout of GRP78 leads to embryonic lethality which means that high affinity GRP78 specific antibody producing B cells would be subject to clonal deletion or clonal anergy during normal B cell development. In addition, GRP78 is highly conserved among vertebrate species. Human GRP78 is 99% identical to primate GRP78 and 98% identical to rodent GRP78. This makes it more difficult to generate high affinity human GRP78 specific antibodies by animal immunization. Overall, the production of antibodies specific to GRP78 requires careful consideration to ensure the generation of reliable and specific antibodies for research or therapeutic applications.

### SUMMARY OF THE INVENTION

Embodiments of the disclosure relate to anti-GRP78 antibodies that is specific for GRP78, as well as methods of using such antibodies in treatment of cancers. By specifically binding GRP78, antibodies as disclosed herein are able to be used to treat cancers that is caused by or related to GRP78 activity or signaling, including lung cancer, breast cancer, prostate cancer, colorectal cancer, gastric cancer, pancreatic cancer, and ovarian cancer.

Accordingly, the present disclosure provides an isolated antibody or antigen-binding fragment thereof that is specific for an epitope in GRP78; wherein the isolated antibody or antigen-binding fragment thereof comprises complementarity determining regions (CDRs) of a heavy chain variable region or CDRs of a light chain variable region, wherein the CDRs of the heavy chain variable region comprise:
CDRH1 having the amino acid sequence of SEQ ID NO: 1 or the amino acid sequence with at least about 90%, 91%, 92%, 93%, 94%, 95%, 96%, 97%, 98%, or 99% identity to SEQ ID NO: 1, CDRH2 having the amino acid sequence of SEQ ID NO: 2 or the amino acid sequence with at least about 90%, 91%, 92%, 93%, 94%, 95%, 96%, 97%, 98%, or 99% identity to SEQ ID NO: 2, and CDRH3 having the amino acid sequence of SEQ ID NO: 3 or the amino acid sequence with at least about 90%, 91%, 92%, 93%, 94%, 95%, 96%, 97%, 98%, or 99% identity to SEQ ID NO: 3; or
wherein the CDRs of the light chain variable region comprise:
CDRL1 having the amino acid sequence of SEQ ID NO: 4 or the amino acid sequence with at least about 90%, 91%, 92%, 93%, 94%, 95%, 96%, 97%, 98%, or 99% identity to SEQ ID NO: 4, CDRL2 having the amino acid sequence of SEQ ID NO: 5 or the amino acid sequence with at least about 90%, 91%, 92%, 93%, 94%, 95%, 96%, 97%, 98%, or 99% identity to SEQ ID NO: 5, and CDRL3 having the amino acid sequence of SEQ ID NO: 6 or the amino acid sequence with at least about 90%, 91%, 92%, 93%, 94%, 95%, 96%, 97%, 98%, or 99% identity to SEQ ID NO: 6.

In some embodiments, the heavy chain variable region comprises the amino acid sequence of SEQ ID NO: 7 or the amino acid sequence with at least about 90%, 91%, 92%, 93%, 94%, 95%, 96%, 97%, 98%, or 99% identity to SEQ ID NO: 7; or the light chain variable region comprises the amino acid sequence of SEQ ID NO: 8 or the amino acid sequence with at least about 90%, 91%, 92%, 93%, 94%, 95%, 96%, 97%, 98%, or 99% identity to SEQ ID NO: 8. In some embodiments, the heavy chain variable region comprises the amino acid sequence of SEQ ID NO: 7, and the light chain variable region comprises the amino acid sequence of SEQ ID NO: 8.

In some embodiments, the isolated antibody or antigen-binding fragment thereof is an Fab fragment, an F(ab′)₂ fragment, an ScFv fragment, a monoclonal antibody, a chimeric antibody, a nanobody, a humanized antibody or a human antibody. In some embodiments, the isolated antibody or antigen-binding fragment thereof is an ScFv fragment comprising the heavy chain variable region having the amino acid sequence of SEQ ID NO: 7 or the amino acid sequence with at least about 90%, 91%, 92%, 93%, 94%, 95%, 96%, 97%, 98%, or 99% identity to SEQ ID NO: 7, the light chain variable region having the amino acid sequence of SEQ ID NO: 8 or the amino acid sequence with at least about 90%, 91%, 92%, 93%, 94%, 95%, 96%, 97%, 98%, or 99% identity to SEQ ID NO: 8, and a linker linking the heaving chain variable region and the light chain variable region. In some embodiments, the linker comprises the amino acid sequence of SEQ ID NO: 21 or 12 or the amino acid sequence with at least about 95% identity to SEQ ID NO: 21 or 12.

In some embodiments, the isolated antibody or antigen-binding fragment thereof is linked to a second specific binding domain for a second target. In some embodiments, the second specific binding domain comprises a T-cell targeting domain. In some embodiments, the second target comprises CD3. In some embodiments, the second specific binding domain comprises the amino acid sequence of SEQ ID NO: 19 or the amino acid sequence with at least about 90%, 91%, 92%, 93%, 94%, 95%, 96%, 97%, 98%, or 99% identity to SEQ ID NO: 19.

In some embodiments, the isolated antibody or antigen-binding fragment thereof is expressed on a surface of a cell. In some embodiments, the cell is an immune cell. In some embodiments, the cell is a T cell.

In some embodiments, the isolated antibody or antigen-binding fragment thereof is conjugated with a therapeutic agent. In some embodiments, the isolated antibody or antigen-binding fragment thereof is covalently linked to a therapeutic agent.

One aspect of the disclosure relates to an immunoconjugate. An immunoconjugate in accordance with one embodiment of the disclosure includes:
the isolated antibody or antigen-binding fragment thereof as disclosed herein, and a therapeutic agent or a label.

In some embodiments of the disclosure, the immunoconjugate has the formula of Ab-(L-D)m,
wherein Ab represents the isolated antibody or antigen-binding fragment thereof as disclosed herein,
L represents a linker or a direct bond,
D represents the therapeutic agent or the label, and
m represents an integer from 1 to 15, 2 to 14, 3 to 13, 4 to 12, 5 to 11, 6 to 10, 7 to 9, or 8.

Embodiments of the disclosure relate to antibody-drug conjugates containing GRP78 antibodies and their uses in therapy. The expression of cell surface GRP78 is associated with cancer and contributes to the maintenance and progression of the disease. Therefore, ADCs based on antibodies against GRP78 can be useful diagnostic and/or treatment agents.

However, the fast internalization or lacking ADCC activity of therapeutic antibody might result in antibody ineffective as well as resistance. Therefore, there is a need to enhance the therapeutic efficacy of anti-GRP78 based therapeutics. One approach is to conjugate a payload with an anti-GRP78 antibody (i.e., an antibody-drug conjugate). By conjugating anti-GRP78 antibodies to payloads (i.e., ADCs), embodiments of the disclosure are more potent than the naked anti-GRP78 antibodies, thereby enabling one to use less antibodies.

In some embodiments of the present disclosure, the anti-GRP78 antibody or the antigen-binding fragment thereof may be used as antibody-drug conjugates (ADCs), which can specifically target GRP78. That is, the present disclosure also provides an immunoconjugate, including the aforementioned isolated anti-GRP78 antibody or the antigen-binding fragment, and therapeutic agent conjugated with the anti-GRP78 antibody or the antigen-binding fragment thereof. The therapeutic agent or payload can be any that are commonly used in ADCs.

In some embodiments of the disclosure, the therapeutic agent represents a cytostatic or cytotoxic agent or an isotope-chelating agent with corresponding radioisotopes. Examples of the cytostatic or cytotoxic agent include, without limitation, antimetabolites (e.g., fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed); alkylating agents (e.g., cmelphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, carboplatin, nedaplatin, oxaliplatin, satraplatin, or triplatin); DNA minor groove alkylating agents (e.g., duocarmycins such as CC-1065, and any analogs or derivatives thereof; pyrrolobenzodiazapenes, or any analogs or derivatives thereof); anthracyclines (e.g., daunorubicin, doxorubicin, epirubicin, idarubicin, or valrubicin); antibiotics (e.g., dactinomycin, bleomycin, mithramycin, anthramycin, streptozotocin, gramicidin D, mitomycins (e.g., mitomycin C); calicheamicins; antimitotic agents (including, e.g., maytansinoids (such as DM1, DM3, and DM4), auristatins (including, e.g., monomethyl auristatin E (MMAE) and monomethyl auristatin F (MMAF)), dolastatins, cryptophycins, vinca alkaloids (e.g., vincristine, vinblastine, vindesine, vinorelbine), taxanes (e.g., paclitaxel, docetaxel, or a novel taxane), tubulysins, and colchicines); topoisomerase inhibitors (e.g., irinotecan, topotecan, SN38, exatecan, deruxtecan, camptothecin, etoposide, teniposide, amsacrine, or mitoxantrone); HDAC inhibitor (e.g., vorinostat, romidepsin, chidamide, panobinostat, or belinostat); proteasome inhibitors (e.g., peptidyl boronic acids); as well as radioisotopes such as At²¹¹, I¹³¹, I¹²⁵, Y⁹⁰, Re¹⁸⁶, Re¹⁸⁸, Sm¹⁵³, Bi²¹² or ²¹³, p³² and radioactive isotopes of Lu including Lu¹⁷⁷. Examples of the isotope-chelating agents include, without limitation, ethylenediaminetetraacetic acid (EDTA), diethylenetriamine-N,N,N′,N″,N″-pentaacetate (DTPA), 1,4,7,10-tetraazacyclododecane-N,N′,N″,N′″-tetraacetate (DOTA), 1,4,7,10-tetrakis(2-hydroxypropyl)-1,4,7,10-tetraazacyclododecane (THP), triethylenetetraamine-N,N,N′,N″,N′″,N′″-hexaacetate (TTHA), 1,4,7,10-tetraazacyclododecane-N,N′,N″,N′″-tetrakis(methylenephosphonate) (DOTP), and mercaptoacetyltriglycine (MAG3).

The present disclosure further provides a vector encoding the isolated antibody or antigen-binding fragment thereof.

The present disclosure also provides a genetically engineered cell containing the vector or expressing the isolated antibody or antigen-binding fragment thereof.

In some embodiments, the genetically engineered cell is an immune cell or a stem cell. In some embodiments, the genetically engineered cell is a T cell.

The present disclosure further provides a bispecific antibody comprising:
(i) an antigen-binding region comprising the isolated antibody or antigen-binding fragment thereof; and
(ii) a T-cell targeting domain linked to the antigen-binding region.

In some embodiments, the T-cell targeting domain is specific for CD3. In some embodiments, the T-cell targeting domain comprises CDRs of a heavy chain variable region and CDRs of a light chain variable region, wherein the heavy-chain variable region comprises HCDR1 having the amino acid sequence of SEQ ID NO: 13 or the amino acid sequence with at least about 90%, 91%, 92%, 93%, 94%, 95%, 96%, 97%, 98%, or 99% identity to SEQ ID NO: 13, HCDR2 having the amino acid sequence of SEQ ID NO: 14 or the amino acid sequence with at least about 90%, 91%, 92%, 93%, 94%, 95%, 96%, 97%, 98%, or 99% identity to SEQ ID NO: 14, and HCDR3 having the amino acid sequence of SEQ ID NO: 15 or the amino acid sequence with at least about 90%, 91%, 92%, 93%, 94%, 95%, 96%, 97%, 98%, or 99% identity to SEQ ID NO: 15; and the light-chain variable region comprises LCDR1 having the amino acid sequence of SEQ ID NO: 16 or the amino acid sequence with at least about 90%, 91%, 92%, 93%, 94%, 95%, 96%, 97%, 98%, or 99% identity to SEQ ID NO: 16, LCDR2 having the amino acid sequence of SEQ ID NO: 17 or the amino acid sequence with at least about 90%, 91%, 92%, 93%, 94%, 95%, 96%, 97%, 98%, or 99% identity to SEQ ID NO: 17, and LCDR3 having the amino acid sequence of SEQ ID NO: 18 or the amino acid sequence with at least about 90%, 91%, 92%, 93%, 94%, 95%, 96%, 97%, 98%, or 99% identity to SEQ ID NO: 18. In some embodiments, the T-cell targeting domain comprises the amino acid sequence of SEQ ID NO: 19 or the amino acid sequence with at least about 90%, 91%, 92%, 93%, 94%, 95%, 96%, 97%, 98%, or 99% identity to SEQ ID NO: 19.

In some embodiments, the antigen-binding region is an ScFv fragment comprising the heavy chain variable region having the amino acid sequence of SEQ ID NO: 7 or the amino acid sequence with at least about 90%, 91%, 92%, 93%, 94%, 95%, 96%, 97%, 98%, or 99% identity to SEQ ID NO: 7, the light chain variable region comprises the amino acid sequence of SEQ ID NO: 8 or the amino acid sequence with at least about 90%, 91%, 92%, 93%, 94%, 95%, 96%, 97%, 98%, or 99% identity to SEQ ID NO: 8, and a linker linking the heaving chain variable region and the light chain variable region. In some embodiments, the linker comprises the amino acid sequence of SEQ ID NO: 21 or 12 or the amino acid sequence with at least about 90%, 91%, 92%, 93%, 94%, 95%, 96%, 97%, 98%, or 99% identity to SEQ ID NO: 21 or 12.

In some embodiments, the antigen-binding region and the T-cell targeting domain is linked by a constant region of an antibody. In some embodiments, the bispecific antibody comprises the amino acid sequence of SEQ ID NO: 22 or 20 or the amino acid sequence with at least about 90%, 91%, 92%, 93%, 94%, 95%, 96%, 97%, 98%, or 99% identity to SEQ ID NO: 22 or 20.

In some embodiments, the bispecific antibody comprises a first heavy chain having the amino acid sequence of SEQ ID NO: 9 or the amino acid sequence with at least about 90%, 91%, 92%, 93%, 94%, 95%, 96%, 97%, 98%, or 99% identity to SEQ ID NO: 9, a second heavy chain having the amino acid sequence of SEQ ID NO: 10 or the amino acid sequence with at least about 90%, 91%, 92%, 93%, 94%, 95%, 96%, 97%, 98%, or 99% identity to SEQ ID NO: 10, and a light chain having the amino acid sequence of SEQ ID NO: 11 or the amino acid sequence with at least about 90%, 91%, 92%, 93%, 94%, 95%, 96%, 97%, 98%, or 99% identity to SEQ ID NO: 11.

The present disclosure also provides a pharmaceutical composition, comprising an effective amount of the isolated antibody or antigen-binding fragment thereof, the genetically engineered cell, or the bispecific antibody; and optionally a pharmaceutically acceptable carrier.

The present disclosure further provides a pharmaceutical composition for use in treating, prophylactic treating and/or preventing a disease and/or disorder caused by or related to GRP78 activity or signaling in a subject in need thereof, comprising an effective amount of the isolated antibody or antigen-binding fragment thereof, the genetically engineered cell, or the bispecific antibody; and optionally a pharmaceutically acceptable carrier. Alternatively, the present disclosure further provides use of a pharmaceutical composition in the manufacture of a medicament for treating, prophylactic treating and/or preventing a disease and/or disorder caused by or related to GRP78 activity or signaling in a subject in need thereof, wherein the pharmaceutical composition comprises an effective amount of the isolated antibody or antigen-binding fragment thereof or the genetically engineered cell, and optionally a pharmaceutically acceptable carrier. Alternatively, the present disclosure provides a method for treating, prophylactic treating and/or preventing a disease and/or disorder caused by or related to GRP78 activity or signaling in a subject in need thereof, comprising administering the pharmaceutical composition to the subject in need thereof.

In some embodiments, the disease is a cancer. In some embodiments, the cancer is a solid cancer. In some embodiments, the cancer is lung cancer, breast cancer, prostate cancer, colorectal cancer, gastric cancer, pancreatic cancer, ovarian cancer, hepatocellular carcinoma, renal cell carcinoma, testicular cancer, melanoma, leukemia, or papillary thyroid cancer.

In some embodiments, the cancer is lung cancer, breast cancer, prostate cancer, colorectal cancer, gastric cancer, pancreatic cancer, or ovarian cancer.

The present disclosure further provides a method for detecting expression of GRP78 or a cancer, comprising contacting the sample with the isolated antibody or antigen-binding fragment thereof.

The present disclosure also provides a kit for detecting GRP78 or a cancer in a sample, comprising the isolated antibody or antigen-binding fragment thereof.

### BRIEF DESCRIPTION OF DRAWINGS

FIG. 1 shows the workflow for obtaining clones of antibodies against GRP78.

FIG. 2 shows the binding affinity of the anti-GRP78 antibody G2D03 by ELISA assay, in comparison to the conventional anti-GRP78 antibody C38.

FIG. 3 shows kinetics binding affinity assay of the anti-GRP78 antibody G2D03 by Biacore.

FIGS. 4A to 4D show FACS analysis of GRP78 expression on the surfaces of several ovarian cancer cells by using the anti-GRP78 antibody G2D03 or the conventional anti-GRP78 antibody C38. The anti-GRP78 antibody G2D03 (30 μg/mL) displayed binding to the cell surfaces of OVCAR3 (FIG. 4B), OV90 (FIG. 4B), SKOV3.ip1 (FIG. 4D) cell lines. The conventional anti-GRP78 antibody C38 (30 μg/mL) displayed binding to the cell surfaces of OVCAR3 (FIG. 4A).

FIG. 5 shows the stress-Induced overexpression of GRP78 on the surface of OVCAR3 ovarian cancer cell.

FIG. 6 shows a schematic view of BsAb T cell-engager antibody G2D03 BsAb that bind specifically to GRP78 and CD3.

FIG. 7 shows the result of NK cell activation by G2D03 mAb.

FIG. 8 shows the result of T cell activation by G2D03 BsAb.

FIGS. 9A, 9B and 9C show the T cell activation in the target cell-dependent manner by the anti-GRP78 BsAb T cell-engager antibody, G2D03 BsAb.

FIGS. 10A to 10D shows T Cell-mediated Cytotoxicity Assays of G2D03 BsAb by high content screening assay.

### DETAILED DESCRIPTION OF THE INVENTION

It is understood that this disclosure is not limited to the particular materials and methods described herein. It is also to be understood that the terminology used herein is for the purpose of describing particular embodiments and is not intended to limit the scope of the present invention, which will be limited only by the appended claims. Unless otherwise defined, scientific and technical terms used herein shall have the meanings that are commonly understood by those of ordinary skill in the art. Furthermore, unless otherwise required by context, singular terms shall include pluralities and plural terms shall include the singular. Generally, nomenclatures used in connection with, and techniques of, cell and tissue culture, molecular biology, and protein and oligo- or polynucleotide chemistry and hybridization described herein are those well-known and commonly used in the art.

The practice of the present disclosure may employ technologies comprising conventional techniques of cell biology, cell culture, antibody technology, and genetic engineering, which are within the ordinary skills of the art. Such techniques are explained fully in the literature.

As utilized in accordance with the present disclosure, the following terms, unless otherwise indicated, shall be understood to have the following meanings: The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

It must be noted that, as used in this specification and the appended claims, the singular forms “a,” “an,” and “the” include plural referents unless the content clearly dictates otherwise.

The term “antibody”, as used herein, means any antigen-binding molecule or molecular complex comprising at least one CDR that specifically binds to or interacts with a particular antigen (e.g., GRP78). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a V_{H} and a heavy chain constant region. The heavy chain constant region comprises three domains, C_{H1}, C_{H2} and C_{H3}. Each light chain comprises a V_{L} and a light chain constant region. The light chain constant region comprises one domain (C_{L1}). The V_{H} and V_{L} can be further subdivided into regions of hypervariability, termed CDRs, interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-GRP78 antibody (or antigen-binding portion thereof) may be identical to the human germline sequences, or may be naturally or artificially modified. An amino acid consensus sequence may be defined based on a side-by-side analysis of two or more CDRs.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

As used herein, the term “being specific for” or “binding specifically to” means that an antibody does not cross react to a significant extent with other epitopes.

As used herein, the term “epitope” refers to the site on the antigen to which an antibody binds.

As used herein, the term “complementarity determining region (CDR)” refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

As applied to polypeptides, the term “substantial similarity” or “substantially similar” means that two peptide sequences, when optimally aligned, such as by the programs GAP or BESTFIT using default gap weights, share at least 90%, 91%, 92%, 93%, 94%, 95%, 96%, 97%, 98% or 99% sequence identity. In some embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

The term “monoclonal antibody (mAb)” as used herein is not limited to antibodies produced through hybridoma technology. A monoclonal antibody is derived from a single clone, including any eukaryotic, prokaryotic, or phage clone, by any means available or known in the art.

The term “chimeric” antibody as used herein refers to an antibody having variable sequences derived from a non-human immunoglobulin and human immunoglobulin constant regions, typically chosen from a human immunoglobulin template.

As used herein, the term “bispecific” as used herein means the polypeptide is capable of specifically binding at least two target entities. The polypeptide is a bispecific antibody (numerous examples of which are described in detail below). Thus, the first and/or second binding domains may be selected from the group consisting of antibodies and antigen-binding fragments thereof. By “an antibody or an antigen-binding fragment thereof” we include substantially intact antibody molecules, as well as chimeric antibodies, humanized antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include Fv fragments (e.g. single chain Fv and disulphide-bonded Fv) and Fab-like fragments (e.g. Fab fragments, Fab′ fragments and F(ab)₂ fragments). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

“Humanized” forms of non-human antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence.

As used herein, the term “nanobody” refers to an antibody comprising the small single variable domain (VHH of antibodies obtained from camelids and dromedaries. Antibody proteins obtained from members of the camel and dromedary (Camelus baclrianus and Calelus dromaderius) family including new world members such as llama species (Lama pacos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

As used in the present disclosure, the term “therapeutic agent” means any compound, substance, drug, drug or active ingredient having a therapeutic or pharmacological effect that is suitable for administration to a mammal, for example a human.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “T cell” includes CD4⁺ T cells and CD8⁺ T cells. The term T cell also includes T helper 1 type T cells, T helper 2 type T cells, T helper 17 type T cells and inhibitory T cells.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

As used herein, the term “immunoconjugate” refers to an antigen-binding protein, e.g., an antibody or antigen-binding fragment, which is chemically or biologically linked to a radioactive agent, a cytokine, an interferon, a target or reporter moiety, an enzyme, a peptide or protein or a therapeutic agent. The antigen-binding protein may be linked to the radioactive agent, cytokine, interferon, target or reporter moiety, enzyme, peptide or therapeutic agent at any location along the molecule so long as it is able to bind its target (GRP78). Examples of immunoconjugates include antibody-drug conjugates and antibody-toxin fusion proteins. In one embodiment of the invention, the agent may be a second, different antibody that binds specifically to GRP78. The type of therapeutic moiety that may be conjugated to the anti-GRP78 protein (e.g., antibody or fragment) will take into account the condition to be treated and the desired therapeutic effect to be achieved.

As used herein, the term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “genetically engineered” or “genetic engineering” of cells means manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

As used in the present disclosure, the term “pharmaceutical composition” means a mixture containing therapeutics administered to a mammal, for example a human, for preventing, treating, or eliminating a particular disease or pathological condition that the mammal suffers.

As used herein, the term “therapeutically effective amount” or “effective amount” refers to the amount of an antibody that, when administered to a mammal or other subject for treating a disease, is sufficient to effect such treatment for the disease.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

“Cancer,” “tumor,” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

As used herein, the term “sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

The present disclosure provides an antibody that is specific for and has high affinities for an epitope in GRP78, such as human GRP78. The anti-GRP78 antibody or an antigen-binding fragment thereof can deliver therapeutic benefits to a subject. The anti-GRP78 antibody or the antigen-binding fragment thereof according to the disclosure can be used as therapeutics for treating and/or diagnosing a variety of disorders mediated by GRP78, which are more fully described herein.

The antibody or the antigen-binding fragment thereof according to embodiments of the disclosure can be full-length (for example, having a heavy chain constant region selected from the group consisting of IgG1, IgG2 and IgG4 isoforms, and a light chain constant region selected from the group consisting of K and A isotypes), or may comprise only an antigen-binding portion (for example, a Fab, F(ab′)₂, or scFv fragment), and may be modified to affect functionalities as needed.

Non-limiting examples of an antigen-binding fragment includes: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody typically comprises at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

As with a full antibody molecule, an antigen-binding fragment may be monospecific or multi-specific (e.g., bispecific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bispecific antibody formats disclosed herein, may be adapted for use in the context of an antigen-binding fragment of an antibody of the present invention using routine techniques available in the art.

Particularly, isolated antibody or antigen-binding fragment thereof that is specific for an epitope in GRP78 comprises CDRs of a heavy chain variable region and CDRs of a light chain variable region,
wherein the CDRs of the heavy chain variable region comprise:
CDRH1 having the amino acid sequence of SEQ ID NO: 1 or a substantially similar sequence thereof, CDRH2 having the amino acid sequence of SEQ ID NO: 2 or a substantially similar sequence thereof, and CDRH3 having the amino acid sequence of SEQ ID NO: 3 or a substantially similar sequence thereof; and
wherein the CDRs of the light chain variable region comprise:
CDRL1 having the amino acid sequence of SEQ ID NO: 4 or a substantially similar sequence thereof, CDRL2 having the amino acid sequence of SEQ ID NO: 5 or a substantially similar sequence thereof, and CDRL3 having the amino acid sequence of SEQ ID NO: 6 or a substantially similar sequence thereof.

The sequences in the disclosure are shown in Table 1.

TABLE 1

| | | SEQ |
|---|---|---|
| | | ID |
| | Sequence | NO. |
| G2D03_ | GFTFSQYAMA | 1 |
| CDRH1 | | |
| G2D03_ | GISPYGSNTRYADSVKG | 2 |
| CDRH2 | | |
| G2D03_ | NGGYSYTWFDL | 3 |
| CDRH3 | | |
| G2D03_ | RASQSISSDLA | 4 |
| CDRL1 | | |
| G2D03_ | WASKLQS | 5 |
| CDRL2 | | |
| G2D03_ | MQMYNTPPT | 6 |
| CDRL3 | | |
| G2D03_ | EVQLVESGGGLVQPGGSLRLSCAASGFTFSQYAMAWVRQAPGKGLEWVSGI | 7 |
| VH | SPYGSNTRYADSVKGRFTISRDNSKNTLYLQMNSLRAEDTAVYYCARNGGYSY | |
| | TWFDLWGQGTLVTVSS | |
| G2D03_ | DIQMTQSPSSLSASVGDRVTITCRASQSISSDLAWYQQKPGKAPKLLIYWASKL | 8 |
| VL | QSGVPSRFSGSGSGTDFTLTISSLQPEDFATYYCMQMYNTPPTFGQGTKVEIK | |
| | R | |
| G2D03 | EVQLVESGGGLVQPGGSLRLSCAASGFTFSQYAMAWVRQAPGKGLEWVSGI | 9 |
| BsAb_ | SPYGSNTRYADSVKGRFTISRDNSKNTLYLQMNSLRAEDTAVYYCARNGGYSY | |
| HC | TWFDLWGQGTLVTVSSASTKGPSVFPLAPSSKSTSGGTAALGCLVKDYFPEPV | |
| (knob) | TVSWNSGALTSGVHTFPAVLQSSGLYSLSSVVTVPSSSLGTQTYICNVNHKPSN | |
| | TKVDKKVEPKSCDKTHTCPPCPAPELAGAPSVFLFPPKPKDTLMISRTPEVTCV | |
| | VVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSLW | |
| | CLVKGFYPSDIAVEWESNGQPENNYKTTPPVLDSDGSFFLYSKLTVDKSRWQQ | |
| | GNVFSCSVMHEALHNHYTQKSLSLSPGGGGGSGGGGSGGGGSQVQLVQSG | |
| | AEVKKPGASVKVSCKASGYTFTRYTMHWVRQAPGQGLEWIGYINPSRGYTNY | |
| | NQKFKDKATLTTDKSISTAYMELSRLRSDDTAVYYCARYYDDHYTLDYWGQGT | |
| | LVTVSSGGGGSGGGGSGGGGSEIVLTQSPATLSLSPGERATLSCSASSSVSYM | |
| | NWYQQKPGQAPRRWIYDTSKLASGIPARFSGSGSGTSYTLTISSLEPEDFAVYY | |
| | CQQWSSNPFTFGQGTKVEIKR | |
| G2D03 | EVQLVESGGGLVQPGGSLRLSCAASGFTFSQYAMAWVRQAPGKGLEWVSGI | 10 |
| BsAb_ | SPYGSNTRYADSVKGRFTISRDNSKNTLYLQMNSLRAEDTAVYYCARNGGYSY | |
| HC | TWFDLWGQGTLVTVSSASTKGPSVFPLAPSSKSTSGGTAALGCLVKDYFPEPV | |
| (hole) | TVSWNSGALTSGVHTFPAVLQSSGLYSLSSVVTVPSSSLGTQTYICNVNHKPSN | |
| | TKVDKKVEPKSCDKTHTCPPCPAPELAGAPSVFLFPPKPKDTLMISRTPEVTCV | |
| | VVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSLSC | |
| | AVKGFYPSDIAVEWESNGQPENNYKTTPPVLDSDGSFFLVSKLTVDKSRWQQ | |
| | GNVFSCSVMHEALHNHYTQKSLSLSPG | |
| G2D03 | DIQMTQSPSSLSASVGDRVTITCRASQSISSDLAWYQQKPGKAPKLLIYWASKL | 11 |
| BsAb_ | QSGVPSRFSGSGSGTDFTLTISSLQPEDFATYYCMQMYNTPPTFGQGTKVEIK | |
| LC | RTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRGE | |
| | C | |
| linker | GGGGGSGGGGSGGGGS | 12 |
| linker | GGGGSGGGGSGGGGS | 21 |
| CD3_ | GYTFTRYTMH | 13 |
| CDRH1 | | |
| CD3_ | YINPSRGYTNYNQ | 14 |
| CDRH2 | | |
| CD3_ | YYDDHYTLDY | 15 |
| CDRH3 | | |
| CD3_ | SSSVSYMN | 16 |
| CDRL1 | | |
| CD3_ | DTSKLAS | 17 |
| CDRL2 | | |
| CD3_ | QQWSSNPFT | 18 |
| CDRL3 | | |
| CD3_ | QVQLVQSGAEVKKPGASVKVSCKASGYTFTRYTMHWVRQAPGQGLEWIGYI | 19 |
| scFv | NPSRGYTNYNQKFKDKATLTTDKSISTAYMELSRLRSDDTAVYYCARYYDDHY | |
| | TLDYWGQGTLVTVSSGGGGSGGGGSGGGGSEIVLTQSPATLSLSPGERATLSC | |
| | SASSSVSYMNWYQQKPGQAPRRWIYDTSKLASGIPARFSGSGSGTSYTLTISS | |
| | LEPEDFAVYYCQQWSSNPFTFGQGTKVEIKR | |
| Single | EVQLVESGGGLVQPGGSLRLSCAASGFTFSQYAMAWVRQAPGKGLEWVSGI | 20 |
| chain | SPYGSNTRYADSVKGRFTISRDNSKNTLYLQMNSLRAEDTAVYYCARNGGYSY | |
| (scFv | TWFDLWGQGTLVTVSSGGGGGSGGGGSGGGGS | |
| (GRP78)- | DIQMTQSPSSLSASVGDRVTITCRASQSISSDLAWYQQKPGKAPKLLIYWASKL | |
| scFv | QSGVPSRFSGSGSGTDFTLTISSLQPEDFATYYCMQMYNTPPTFGQGTKVEIK | |
| (CD3)) | RGGGGSGGGGSGGGG | |
| | [symbol] | |
| | [symbol] | |
| | [symbol] | |
| | [symbol] | |
| | [symbol] | |
| Single | EVQLVESGGGLVQPGGSLRLSCAASGFTFSQYAMAWVRQAPGKGLEWVSGI | 22 |
| chain | SPYGSNTRYADSVKGRFTISRDNSKNTLYLQMNSLRAEDTAVYYCARNGGYSY | |
| (scFv | TWFDLWGQGTLVTVSSGGGGSGGGGSGGGGS | |
| (GRP78)- | DIQMTQSPSSLSASVGDRVTITCRASQSISSDLAWYQQKPGKAPKLLIYWASKL | |
| scFv | QSGVPSRFSGSGSGTDFTLTISSLQPEDFATYYCMQMYNTPPTFGQGTKVEIK | |
| (CD3)) | RGGGGSGGGGSGGGGSQVQLVQSGAEVKKPGASVKVSCKASGYTFTRYTM | |
| | HWVRQAPGQGLEWIGYINPSRGYTNYNQKFKDKATLTTDKSISTAYMELSRL | |
| | RSDDTAVYYCARYYDDHYTLDYWGQGTLVTVSSGGGGSGGGGSGGGGSEIV | |
| | LTQSPATLSLSPGERATLSCSASSSVSYMNWYQQKPGQAPRRWIYDTSKLASG | |
| | IPARFSGSGSGTSYTLTISSLEPEDFAVYYCQQWSSNPFTFGQGTKVEIKR | |

* The sequences of both the knob and hole chains contain the immune effector null L235A and G237A mutations (LAGA).

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity.

In some embodiments of the present disclosure, the isolated antibody or antigen-binding fragment thereof includes the heavy chain variable region comprising the amino acid sequence of SEQ ID NO: 7 or a substantially similar sequence thereof; and/or the light chain variable region comprising the amino acid sequence of SEQ ID NO: 8, or a substantially similar sequence thereof.

In some embodiments of the present disclosure, the isolated antibody or antigen-binding fragment thereof includes the heavy chain variable region comprising the amino acid sequence of SEQ ID NO: 7, and the light chain variable region comprising the amino acid sequence of SEQ ID NO: 8.

In some embodiments of the present disclosure, the isolated antibody or antigen-binding fragment thereof is an ScFv fragment comprising the heavy chain variable region having the amino acid sequence of SEQ ID NO: 7 or a substantially similar sequence thereof, the light chain variable region comprises the amino acid sequence of SEQ ID NO: 8 or a substantially similar sequence thereof, and a linker linking the heaving chain variable region and the light chain variable region. In some embodiments of the disclosure, the linker comprises the amino acid sequence of SEQ ID NO: 21 or 12 or a substantially similar sequence thereof.

The isolated antibody or antigen-binding fragment thereof disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes an antibody, and an antigen-binding fragment thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another mammalian germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The isolated antibody or antigen-binding fragment thereof of the present disclosure may be monospecific, bi-specific, or multispecific. Multispecific antibodies may be specific for different epitopes of one target polypeptide or may contain antigen-binding domains specific for more than one target polypeptide. The anti-GRP78 antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present disclosure includes bi-specific antibodies wherein one arm of an immunoglobulin is specific for GRP78 or a fragment thereof, and the other arm of the immunoglobulin is specific for a second target or is conjugated to a therapeutic agent.

In some embodiments of the disclosure, the antibody or antigen-binding fragment thereof is linked to a second binding domain for a second target. For example, the isolated antibody or antigen-binding fragment thereof may be linked to a T-cell targeting domain, in other words, the second specific binding domain comprises a T-cell targeting domain. In some embodiments of the disclosure, the second target comprises CD3. In some further embodiments of the disclosure, the second specific binding domain comprises the amino acid sequence of SEQ ID NO: 19 or a substantially similar sequence thereof.

In some embodiments of the disclosure, the isolated antibody or antigen-binding fragment thereof is in a form of chimeric antigen receptor.

In some embodiments of the present disclosure, the anti-GRP78 antibody or the antigen-binding fragment thereof may be used as antibody-drug conjugates (ADCs), which can specifically target GRP78. That is, the present disclosure also provides an antibody conjugate, including the aforementioned anti-GRP78 antibody or the antigen-binding fragment, and therapeutic agent conjugated with the anti-GRP78 antibody or the antigen-binding fragment thereof. The therapeutic agent or payload can be any that are commonly used in ADCs. In some embodiments, the therapeutic agent or payload is selected from the group consisting of antimetabolites, alkylating agents, alkylating-like agents, DNA minor groove alkylating agents, anthracyclines, antibiotics, calicheamicins, antimitotic agents, topoisomerase inhibitors, HDAC inhibitor, proteasome inhibitors, and radioisotopes. For example, the therapeutic agents or payloads may include mertansine (DM1), monomethyl auristin E (MMAE), seco-DUBA, exactecan, deruxtecan or monomethyl auristatin F (MMAF). The methods for conjugation can be those known in the art.

The genes encoding the heavy and light chains of an antibody of interest can be cloned from a cell, e.g., the genes encoding a monoclonal antibody can be cloned and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)).

An example of a method for manufacturing the antibody or antigen-binding fragment comprises: (a) introducing into a host cell one or more polynucleotides encoding said isolated antibody or antigen-binding fragment; (b) culturing the host cell under conditions favorable to expression of the one or more polynucleotides; and (c) optionally, isolating the antibody or antigen-binding fragment from the host cell and/or a medium in which the host cell is grown.

The isolated antibody or antigen-binding fragment thereof may be encoded in a vector. The present disclosure also provide a vector encoding the anti-GRP78 antibody or the antigen-binding fragment thereof as described herein. In one embodiment, one type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In another aspect, the present disclosure provides a genetically engineered cell expressing the isolated antibody or antigen-binding fragment thereof as described herein or containing the vector as described herein. The genetically engineered cell may be an immune cell, such as a T cell. In one embodiment of the disclosure, the antibody or antigen-binding fragment thereof is expressed on the surface of a cell. Particularly, the cell is a T-cell.

In some embodiments of the disclosure, the isolated antibody or antigen-binding fragment thereof is in a form of chimeric antigen receptor.

The term “chimeric antigen receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains.

The disclosure further provides a bispecific antibody comprising: (i) an antigen-binding region comprising the isolated antibody or antigen-binding fragment thereof as disclosed herein and (ii) a T-cell targeting domain linked to the antigen-binding region.

In some embodiments of the disclosure, the T-cell targeting domain specifically binds to CD3. For example, the T-cell targeting domain comprises CDRs of a heavy chain variable region and CDRs of a light chain variable region, wherein the heavy-chain variable region comprises HCDR1 having the amino acid sequence of SEQ ID NO: 13 or a substantially similar sequence thereof, HCDR2 having the amino acid sequence of SEQ ID NO: 14 or a substantially similar sequence thereof, and HCDR3 having the amino acid sequence of SEQ ID NO: 15 or a substantially similar sequence thereof, and the light-chain variable region comprises LCDR1 having the amino acid sequence of SEQ ID NO: 16 or a substantially similar sequence thereof, LCDR2 having the amino acid sequence of SEQ ID NO: 17 or a substantially similar sequence thereof, and LCDR3 having the amino acid sequence of SEQ ID NO: 18 or a substantially similar sequence thereof. In some embodiments, the T-cell targeting domain may comprise the amino acid sequence of SEQ ID NO: 19 or a substantially similar sequence thereof.

In some embodiments of the disclosure, the antigen-binding region is an ScFv fragment comprising the heavy chain variable region having the amino acid sequence of SEQ ID NO: 7 or a substantially similar sequence thereof, the light chain variable region comprises the amino acid sequence of SEQ ID NO: 8 or a substantially similar sequence thereof, and a linker linking the heaving chain variable region and the light chain variable region. The linker may comprise the amino acid sequence of SEQ ID NO: 21 or 12 or a substantially similar sequence thereof.

In some embodiments of the disclosure, the antigen-binding region and the T-cell targeting domain is linked by a constant region of an antibody. In some embodiments of the disclosure, the bispecific antibody comprises the amino acid sequence of SEQ ID NO: 22 or 20 or a substantially similar sequence thereof.

In some embodiments of the disclosure, the bispecific antibody comprises a first heavy chain having the amino acid sequence of SEQ ID NO: 9 or a substantially similar sequence thereof, a second heavy chain having the amino acid sequence of SEQ ID NO: 10 or a substantially similar sequence thereof, and a light chain having the amino acid sequence of SEQ ID NO: 11 or a substantially similar sequence thereof.

The disclosure further provides pharmaceutical compositions including the isolated antibody or antigen-binding fragment thereof, the genetically engineered cell or the bispecific antibody as described herein. In some embodiments of the disclosure, the pharmaceutical compositions as described herein are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. When an antibody of the present disclosure is used for treating a condition or disease associated with GRP78 in an adult patient, it may be advantageous to intravenously administer the antibody of the present disclosure. Depending on the severity of the condition, the frequency and the duration of the treatment can be adjusted. Effective dosages and schedules for administering the antibody may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In some embodiments of the disclosure, the pharmaceutical composition is for use in treating, prophylactic treating and/or preventing a disease and/or disorder caused by or related to GRP78 activity and/or signaling. Alternatively, the present disclosure further provides use of a pharmaceutical composition in the manufacture of a medicament for treating, prophylactic treating and/or preventing a disease and/or disorder caused by or related to GRP78 activity or signaling in a subject in need thereof, wherein the pharmaceutical composition comprises an effective amount of the isolated antibody or antigen-binding fragment thereof or the genetically engineered cell, and optionally a pharmaceutically acceptable carrier. Alternatively, the present disclosure also provides a method for treating, prophylactic treating and/or preventing a disease and/or disorder caused by or related to GRP78 activity and/or signaling in a subject in need of such treatment, comprising administering to the subject the pharmaceutical composition. In some embodiments, the disease is a cancer, such as solid cancer, including lung cancer, breast cancer, prostate cancer, colorectal cancer, gastric cancer, pancreatic cancer, ovarian cancer, hepatocellular carcinoma, renal cell carcinoma, testicular cancer, melanoma, leukemia, and papillary thyroid cancer.

The present disclosure further provides a method for detecting GRP78 or cancer in a sample, which includes contacting a sample with the isolated antibody or antigen-binding fragment thereof as described herein. The present disclosure also provides a kit for detecting GRP78 in a sample, including the isolated antibody or antigen-binding fragment thereof.

The isolated antibody or antigen-binding fragment thereof as described herein may also be used to detect and/or measure GRP78, or GRP78-expressing cells in a sample, e.g., for diagnostic purposes. For example, an anti-GRP78 antibody, or the antigen binding fragment thereof, may be used to diagnose a condition or disease characterized by aberrant expression (e.g., overexpression, under-expression, lack of expression, etc.) of GRP78. Exemplary diagnostic assays for GRP78 may comprise, e.g., contacting a sample, obtained from a patient, with an anti-GRP78 antibody of the disclosure, wherein the anti-GRP78 antibody is labeled with a detectable label or reporter molecule. Alternatively, an unlabeled anti-GRP78 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure GRP78 in a sample include enzyme-linked immunosorbent assay (ELISA), radioimmunoassay (RIA), and fluorescence-activated cell sorting (FACS).

The following examples are provided to aid those skilled in the art in practicing the present disclosure.

### EXAMPLES

The following examples illustrate the development and use of GRP78-specific antibodies to suppress tumor growth by inducing an anti-GRP78 immune response.

The following examples are provided to aid those skilled in the art in practicing the present disclosure.

### Example 1 Generation of Anti-GRP78 Monoclonal Antibody (G2D03)

1-1 Preparation of Fully Synthetic Human scFv Phage for Bio-Panning

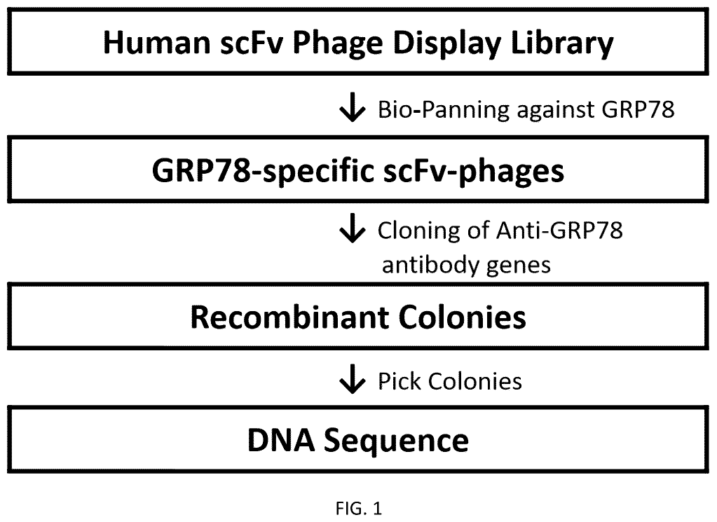

The workflow for obtaining clones of antibodies against GRP78 may be as shown in FIG. 1. The fully synthetic human scFv phage library was inoculated with bacteria into a 2×YT medium containing 100 μg/ml ampicillin and 2% glucose (2YTAG) and incubated by shaking at 37° C. until the OD at 600 nm reaches 0.5. The culture was infected with a helper phage and then cultured without shaking in a 37° C. water bath for 30 min. The cells in the culture were collected and suspended in a 2×YT medium containing 100 μg/ml ampicillin and 25 μg/ml kanamycin (2YTAK) and further incubated with shaking at 30° C. overnight. The supernatant of the culture was collected and mixed with 1/5 volume of PEG/NaCl (20% Polyethylene glycol 8000, 2.5 M NaCl) and stayed at 4° C. for at least one hour. After centrifuging, the pellet was collected and suspended in PBS and spun again to collect the supernatant.

### 1-2 Bio-Panning of ScFv Phages by Using ELISA Method

An ELISA plate (Nunc) was coated with 5 to 25 μg/100 μl of antigen (HSPA5/GRP78 (Active Protein) Human Protein (ORIGENE™, Cat. No. AR03021PU-L)) per well and stayed in sodium bicarbonate buffer (pH 9.6) at 4° C. overnight. The wells were washed 3 times with PBS and blocked with 300 μl of 5% skim milk-containing PBS (MPBS) per well at 37° C. for 2 hours. After being washed 3 times with PBS, 100 μl of phages in 5% MPBS with his-tag-containing fusion protein were added and incubated at 37° C. for 90 min. After being washed 4 to 10 times with 0.05% Tween 20-containing PBS (PBST) and 4 to 10 times with PBS, the phages were eluted by adding 100 μl of 100 mM triethylamine (TEA) and reacted at 37° C. for 20 min. 100 μl of eluted phages were neutralized with 50 μl of 1 M Tris, pH 7.4. 3 mL of TG1 at an exponentially growing stage were added with the eluted phages. The cultures were incubated at 37° C. for 30 min without shaking for infection. The infected TG1 bacteria were added with 20 mL of 2×YT-AG and then incubated at 37° C. overnight.

### 1-3 Preparation of Next Round Phage

The cultures provided by Example 1-2 were spun, collected, and then suspended in 0.5 mL of 2×YT-AG, 15% glycerol. Then, 10 μl of the bacteria were added to 10 mL of 2×YT-AG and the bacteria grew with shaking at 37° C. until the OD at 600 nm reaches 0.5. 10 mL of the culture was infected with M13KO7 helper phage by adding the helper phage at a ratio of 1:20 (M13KO7 helper phage:culture) and the infected culture was incubated without shaking in a 37° C. water bath for 30 min. The cultures were spun to collect the pellet, and the pellet was suspended with 25 mL of 2×YT-AK and then cultured at 30° C. overnight. Further, 25 mL of the overnight culture was spun at 10,000 rpm for 20 min to collect the supernatant, and 1/5 volume (5 mL) PEG/NaCl was added to the supernatant to provide a mixture. The mixture was spun at 10,000 rpm for 20 min and the pellet was collected and suspended in 0.5 mL PBS.

1-4 Screening of Human GRP78 Specific scFv-Phage Clones by ELISA

The suspensions provided by Example 1-3 were spread on the plate, and cultured to obtain individual colonies. The individual colonies thus provided were inoculated into 200 μl of 2×YT-AG 96-well plates and grew with shaking at 37° C. overnight, and then 10 μl of the culture was transferred to a second 96-well plate containing 180 μl of 2×YT-A per well for shaking at 37° C. for 2 hours. Then, 50 μl of 2×YT-A with 2.4×10¹⁰ pfu/mL M13KO7 helper phage was added to each well of the second plate to provide a mixture. The mixture was shaken at 37° C. for 2 hours. 50 μl of 2×YT-AK3 (the kanamycin concentration was 300 μg/mL) was added to the mixture, and then grew with shaking at 30° C. overnight. The culture was added with 50 μl MPBS to provide a phage mixture, and 100 μl of the phage mixture was taken for phage ELISA.

The ELISA plates were coated with 1 μg/mL of protein antigen per well, and then rinsed 3 times with PBS and blocked with 300 μl of 5% MPBS per well at 37° C. for 2 hours. After rinsing a further 3 times with PBS, 100 μl phage mixture as detailed above was added and incubated at 37° C. for 90 min. The phage solution was discarded and the wells were washed 6 times with PBST and 6 times with PBS, and then, an appropriate diluted HRP-anti-M13 antibody in 5% MPBS was added to provide a mixture. The mixture was incubated at 37° C. for 60 min, and then washed 6 times with PBST. The wells were developed with substrate solution (TMB) and the reactions were stopped by adding 100 μl of 1 M Hydrochloric acid. The color turned yellow, and the OD at 650 nm and at 450 nm was assayed.

### 1-5 the Monoclonal Phage Preparation

The clones provided by Example 1-4 were subjected to the procedure as follows. The bacteria were cultured at 37° C. overnight. Thereafter, 100 μl bacteria were added with 2×YT-AG, and the bacteria grew with shaking at 37° C. until the OD at 600 nm reaches 0.5. 10 mL of the culture was infected with M13KO7 helper phage by adding the helper phage at a ratio of 1:20 (M13KO7 helper phage:culture), and the infected culture was incubated without shaking in a 37° C. water bath for 30 min. The cultures were spun to collect the pellet, and the pellet was suspended with 25 mL of 2×YT-AK and then cultured at 30° C. overnight. Further, 25 mL of the overnight culture was spun at 10,000 rpm for 20 min to collect the supernatant, and 1/5 volume (5 ml) PEG/NaCl was added to the supernatant to provide a mixture. The mixture was spun at 10,000 rpm for 20 min, and the pellet was collected and suspended in 0.5 mL PBS.

### 1-6 Expression of Full-Length Antibodies

The genes encoding anti-human GRP78 antibodies were constructed by inserting the VH and VL chains, which come from the scFv phage clones provided by Example 1-5, into an expression vector containing CH and CL chain, respectively. Free-style 293 cells were transfected with the constructed vector. The antibodies were purified by using Protein A Sepharose Fast Flow (GE™ HEALTHCARE, 17-1279-02). After purification, the antibodies were quantified by measuring at OD 280 nm and checked by reducing and non-reducing PAGE. The anti-GRP78 antibody was named as G2D03, and the sequences of the heavy chain variable region, light chain variable region and the CDRs are listed in Table 1.

### Example 2 Binding Affinity of G2D03

### 2-1 the Recombinant GRP78 Protein Immobilization

This ligand has the optimum pH 4.0 condition for direct immobilization by pH scouting test. With amine coupling method, carboxyl groups on the surface of the sensor chip surface are first activated with a mixture of EDC and NHS to give reactive succinimide esters. GRP78 is then passed over the surface and the esters react with primary amine groups to link the ligand covalently to the dextran matrix. The target level of the GRP78 protein is about 300 RU.

### 2-2 the Binding Curve of G2D03 to Recombinant GRP78

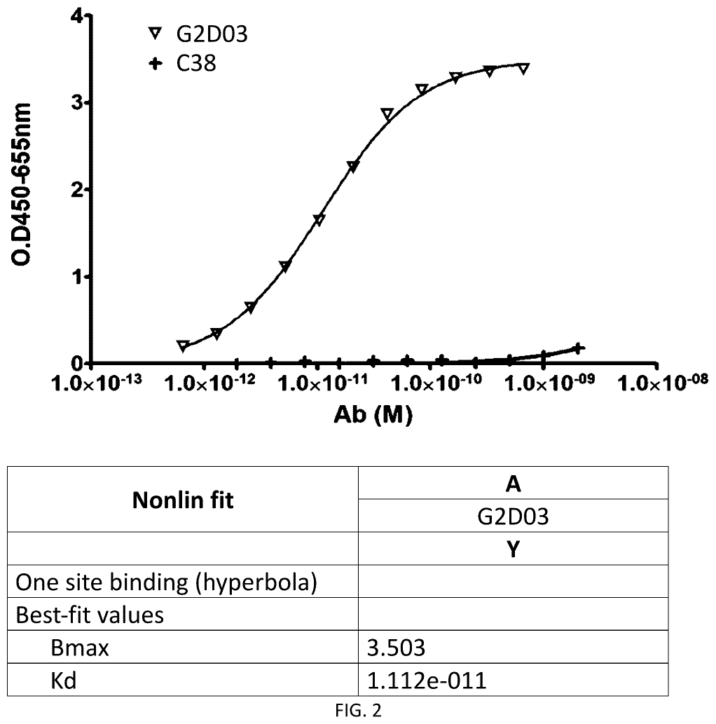

Antigen, GRP78, diluted in sodium carbonate buffer (pH 9.5), was coated on a 96-well plate at 4° C. overnight. After blocking (with 3% BSA), two-fold serial diluted anti-GRP78 antibodies G2D03 or C38 (eBioscience™, Cat. No. 14-9768-82) were added to the wells and incubated at 37° C. for 1 hr. After binding, Goat anti-human IgG(H+L)-HRP (1:15000) and Goat anti-mouse IgG(H+L)-HRP (1:10000) was added to G2D03 and C38 respectively and incubated at 37° C. for 1 hr. Then, 3,3′,5,5′-Tetramethylbenzidine (TMB) substrate was used to develop colors and the reaction was stopped by addition of I N HCl. The extents of antigen-antibody bindings were determined by measuring absorbance at 450-655 nm, using an ELISA reader (BIO-RAD™ iMark microplate absorbance reader). Data was analyzed using GraphPad Prism 5 software and shown in FIG. 2.

### 2-3 Determination of the Binding Kinetics Constant

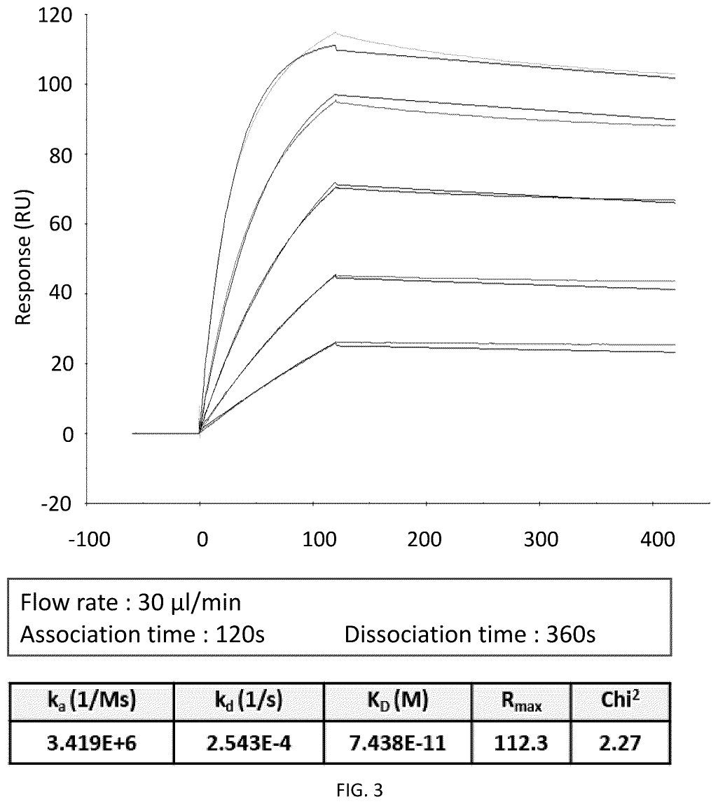

For the multi-cycle kinetics analysis, the chip surface level is 291.2 RU and the analyte concentration is used for the moderate Rₘₐₓ, which is running through the conditions in the assay. The concentrations of the anti-GRP78 antibody G2D03 are: 0.625 nM, 1.25 nM, 2.5 nM, 5 nM and 10 nM. Set the flow rate at 30 μL/min. Association time is 120 seconds. Dissociation time is 360 seconds. Regeneration buffer is performed using 10 mM glycine-HCl pH 1.7. This data sets are analyzed assuming a one-to-one reaction. The on-rate ka is 3.416E+6 (1/Ms) and the off-rate k_{d} is 2.543E-4 (1/s). The KD value is determined to be 7.438E-11 (M) from the ratios of k_{d}/kₐ (FIG. 3). The binding affinity of the anti-GRP78 antibody G2D03 is better than the conventional anti-GRP78 antibody C38.

### Example 3 the Expression of GRP78 in Different Cell Lines

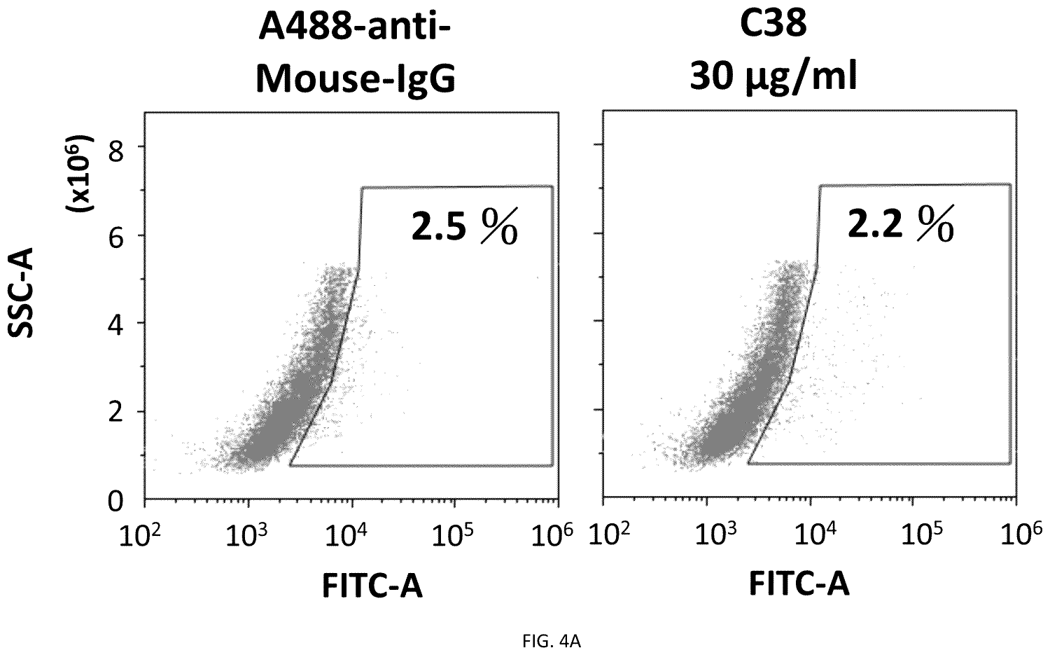

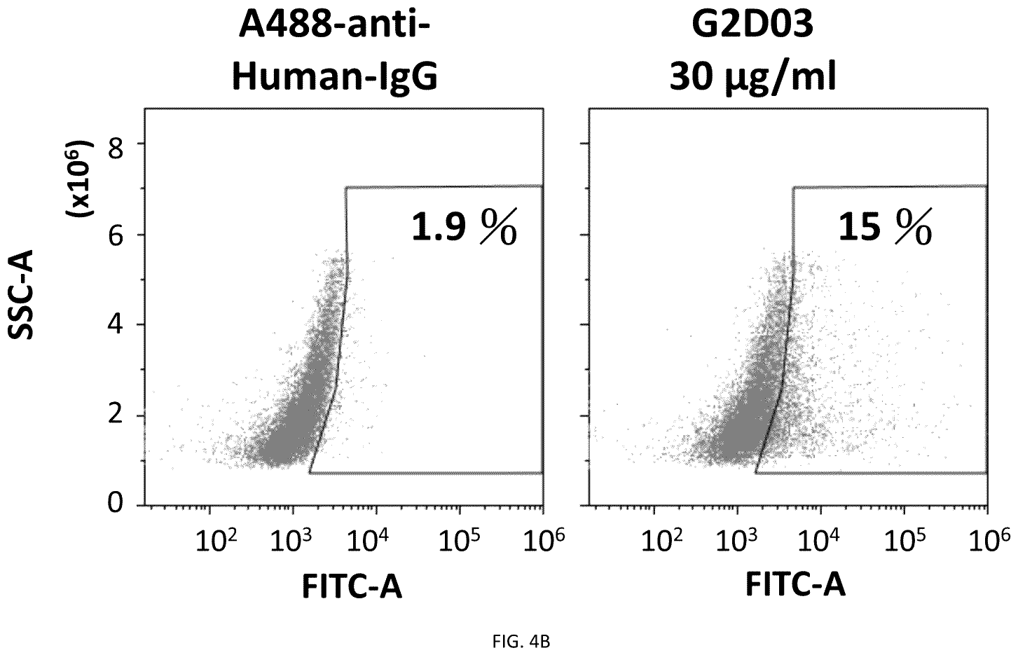

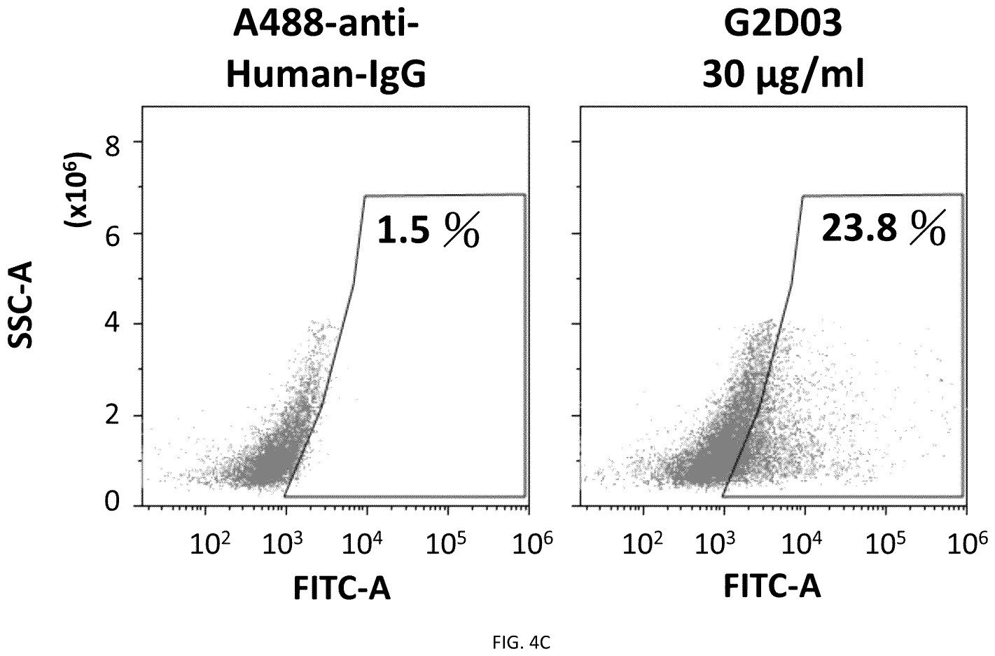

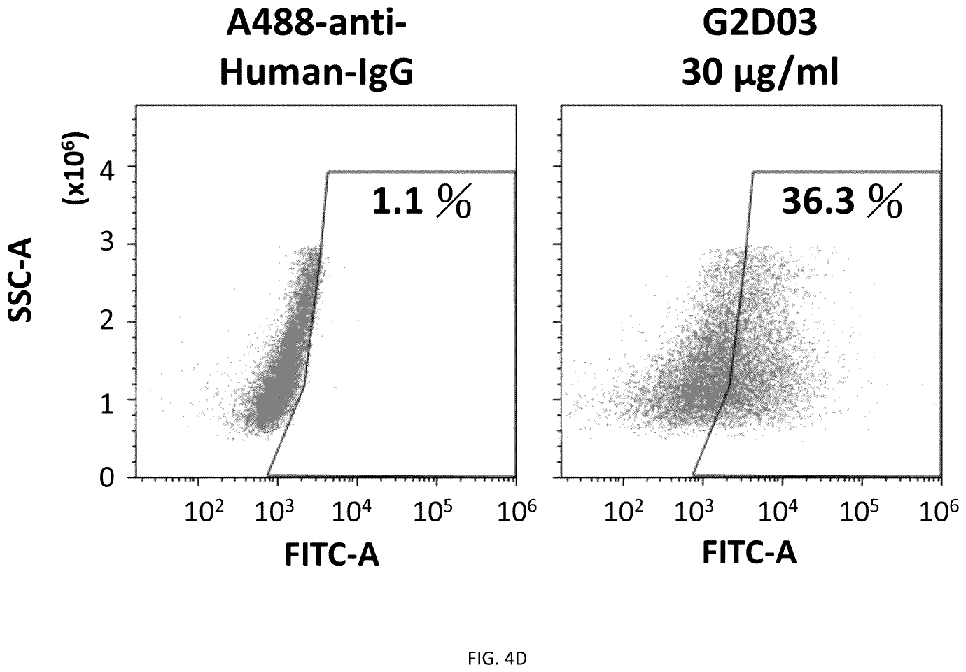

Ovarian cancer cells were analyzed by flow cytometry for cell surface GRP78 expression. The anti-GRP78 antibody G2D03 (30 μg/mL) displayed binding to the cell surface of OVCAR3, OV90, SKOV3, ES2, TOV21G, TOV112D, RMUGS cell lines, as shown in FIGS. 4A to 4D and Table 2. The anti-GRP78 antibody G2D03 (30 μg/mL) displayed binding to the cell surfaces of OVCAR3 in a higher level than the conventional anti-GRP78 antibody C38 (30 μg/mL).

TABLE 2

| | | A488-anti- | C38 | |
|---|---|---|---|---|
| Cancer type | Cell line | mouse-IgG | (30 μg/ml) | |
| OVAIRAN | OVCAR3 | 2.5% | 2.2% | |
| CANCER | | | | |
| | | A488-anti- | | G2D03 |
| | Cell line | human-IgG | | (30 μg/ml) |
| | OVCAR3 | 1.9% | | 15.0% |
| | OV90 | 1.5% | | 23.8% |
| | SKOV3 | 1.1% | | 36.3% |
| | ES2 | 1.2% | | 51.9% |
| | TOV21G | 0.9% | | 62.6% |
| | TOV112D | 1.9% | | 68.3% |
| | RMUGS | 1.8% | | 33.4% |
| PANCREATIC | PL45 | 1.9% | | 21.8% |
| CANCER | ASPC1 | 2.7% | | 23.6% |
| | KLM-1 | 1.1% | | 17.6% |
| | Mia PaCa2 | 4.6% | | 26.9% |
| MELANOMA | G361 | 0.6% | | 53.5% |
| | A375 | 0.9% | | 50.2% |
| LUNG CANCER | NCI-H460 | 1.0% | | 57.2% |
| MULTIPLE | NCI-H929 | 1.7% | | 10.8% |
| MYELOMA | | | | |
| TRIPLE | BT20 | 2.0% | | 28.6% |
| NEGATIVE | HCC1806 | 0.8% | | 78.4% |
| BREAST | MDA- | 2.1% | | 19.6% |
| CANCER | MB-231 | | | |
| LIVER BILE | HuCCT1 | 4.1% | | 18.3% |
| DUCT | | | | |
| CARCINOMA | | | | |

Pancreatic cancer cells were analyzed by flow cytometry for cell surface GRP78 expression. The anti-GRP78 antibody G2D03 (30 μg/mL) displayed binding to the cell surface of PL45, ASPC1, KLM-1, Mia PaCa2 cell lines, as shown in Table 2.

Different type of cancer cells were analyzed by flow cytometry for cell surface GRP78 expression. The anti-GRP78 antibody G2D03 (30 μg/mL) displayed binding to the cell surface of melanoma cells (G361, A375), lung cancer cell (H460), myeloma cell (H929), triple negative breast cancer cells (BT20, HCC1806, MDA-MB-231), liver bile duct carcinoma cell (HuCCT1), as shown in Table 2.

### Example 4 Stress-Induced Overexpression of Surface GRP78 on Ovarian Cancer cell

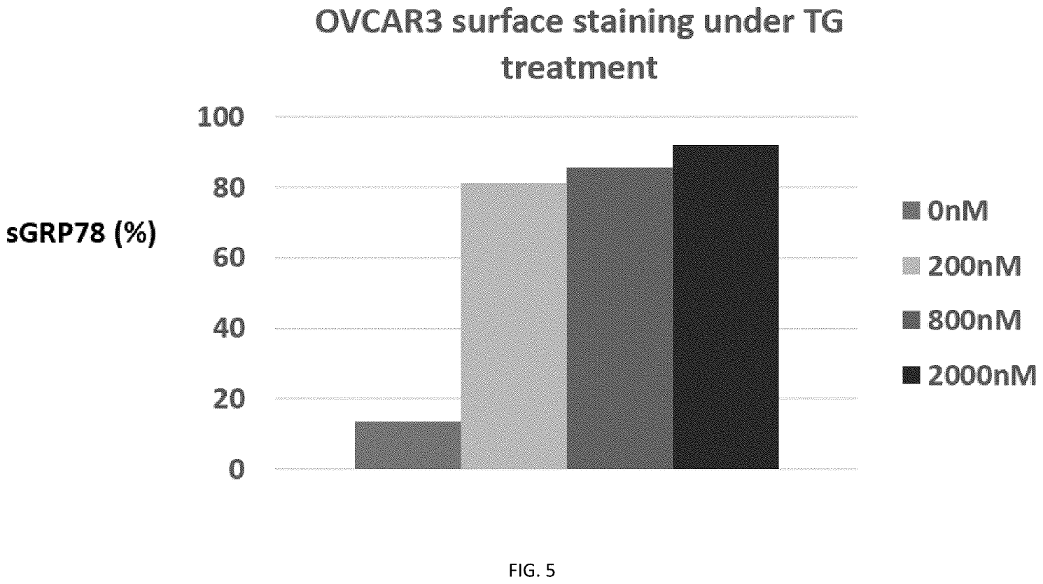

Thapsigargin (TG) is useful in experimentation examining ER stress condition. OVCAR3 cells were treated with different concentrations of thapsigargin for 18 hrs and analyzed by flow cytometry for cell surface GRP78 expression. Results displayed that TG treatment increases surface GRP78 expression on OVCAR3 cells in a dose dependent manner (FIG. 5).

### Example 5 Generation of a Single Domain Bispecific Antibody Specific to GRP78 or CD3

A single domain bispecific antibody was designed to comprise an anti-GRP78 scFv, an anti-CD3 scFv (e.g., SEQ ID NO: 19), and a bridged domain to link the aforementioned anti-GRP78 scFv and anti-CD3 scFv. For example, the single chain bispecific antibody was engineered in the manner of sequentially linked anti-GRP78 scFv, a constant region of IgG, and an anti-CD3 scFv (e.g., SEQ ID NO: 22 or 20).

### Example 6 Generation of G2D03BsAb Specific to GRP78 and CD3

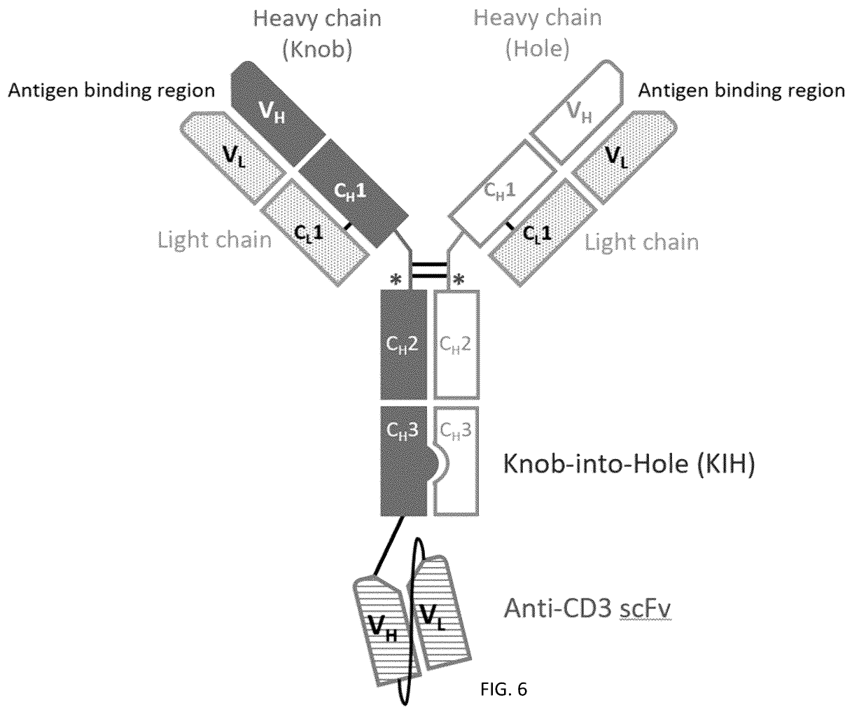

A bispecific antibody was engineered to evaluate for immunotherapy. This involved utilizing the “knob-and-hole” technology, known for its efficient formation of heterodimers. Please refer to U.S. Pat. No. 10,781,265 for more information. In summary, an anti-CD3 scFv was fused separately to the C-terminus of an Fc-hole peptide (G2D03 BsAb_HC(hole)) and/or an Fc-knob peptide (G2D03 BsAb_HC(knob)). The resulting bispecific antibody construct, named G2D03 BsAb, is illustrated in FIG. 6 and listed in Table 1. In this case, the Fc-hole peptide had the amino acid sequence SEQ ID NO: 10, while the Fc-knob peptide had the amino acid sequence SEQ ID NO: 9, and LC peptide had the amino acid sequence SEQ ID NO: 11.

### Example 7 Bioactivity of G2D03 BsAb

7-1 IFN- [symbol] Cytokine Assay

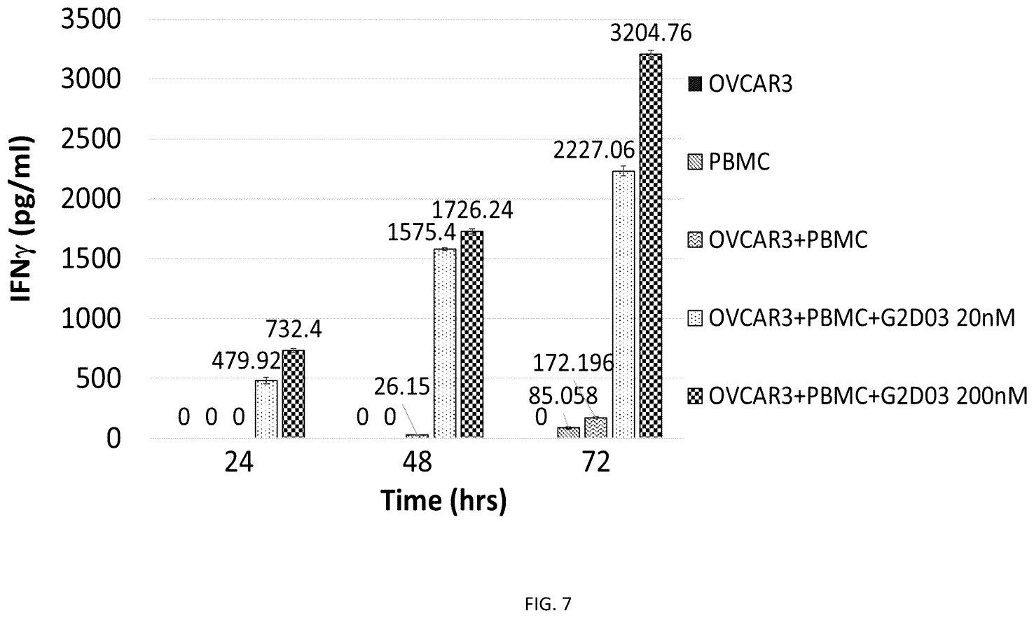

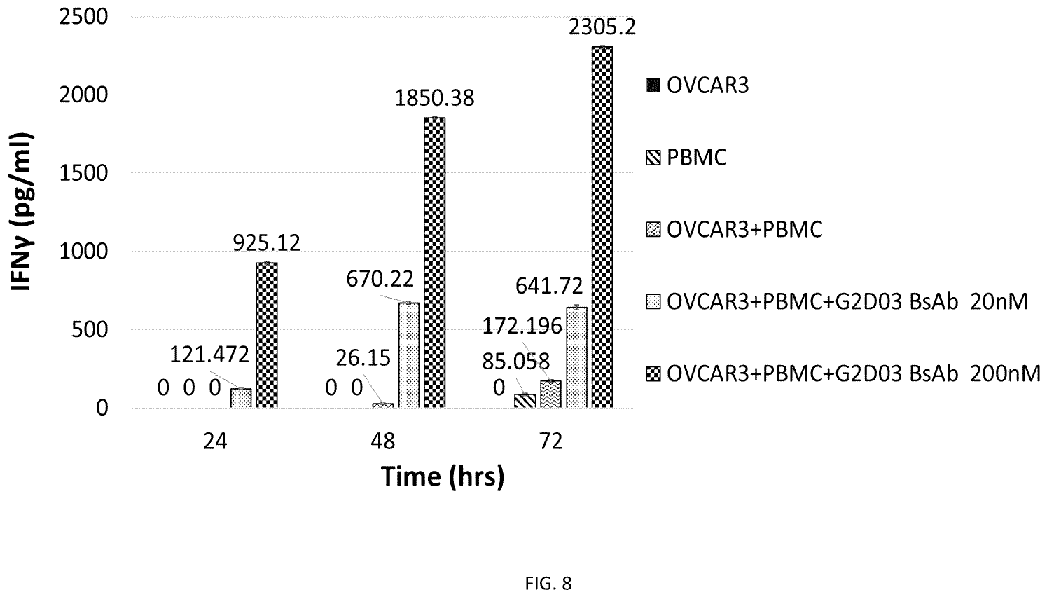

The effector cells, human PBMC cells, were incubated with G2D03 BsAb or G2D03 mAb on GRP78 overexpressing human ovarian carcinoma cell line OVCAR3 at 40:1 E/T ratio for 24, 48, and 72 hrs. Collect supernatants and centrifuge at 800 rpm for 5 min. Supernatants were measured by ELISA MAX Deluxe Set Human IFN-γ. (BIOLEGEND™) [Effector (E) to target (T) ratio, E/T ratio]. The results are shown in FIGS. 7 and 8.

### 7-2 Reporter Assay of Target Cell-Dependent T Cell Activation by G2D03 BsAb

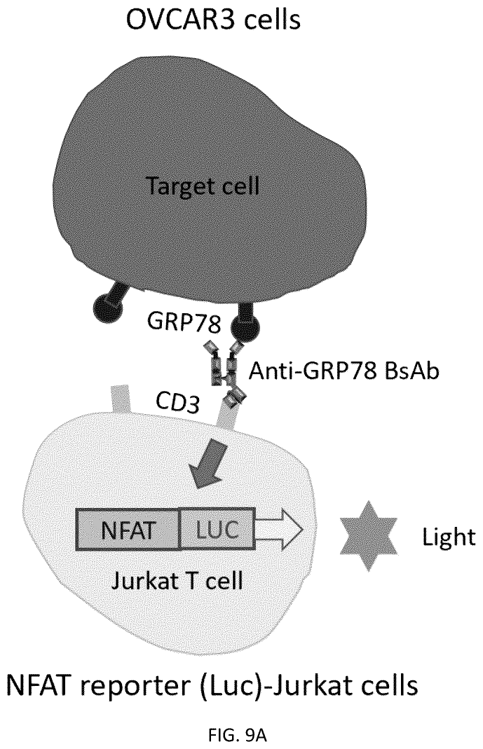

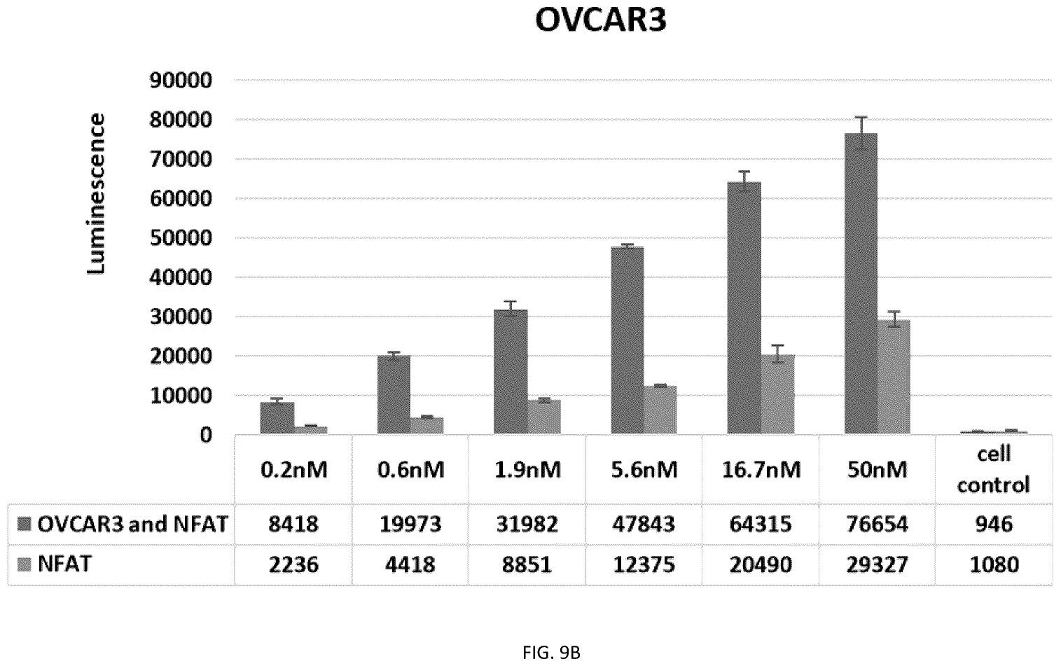

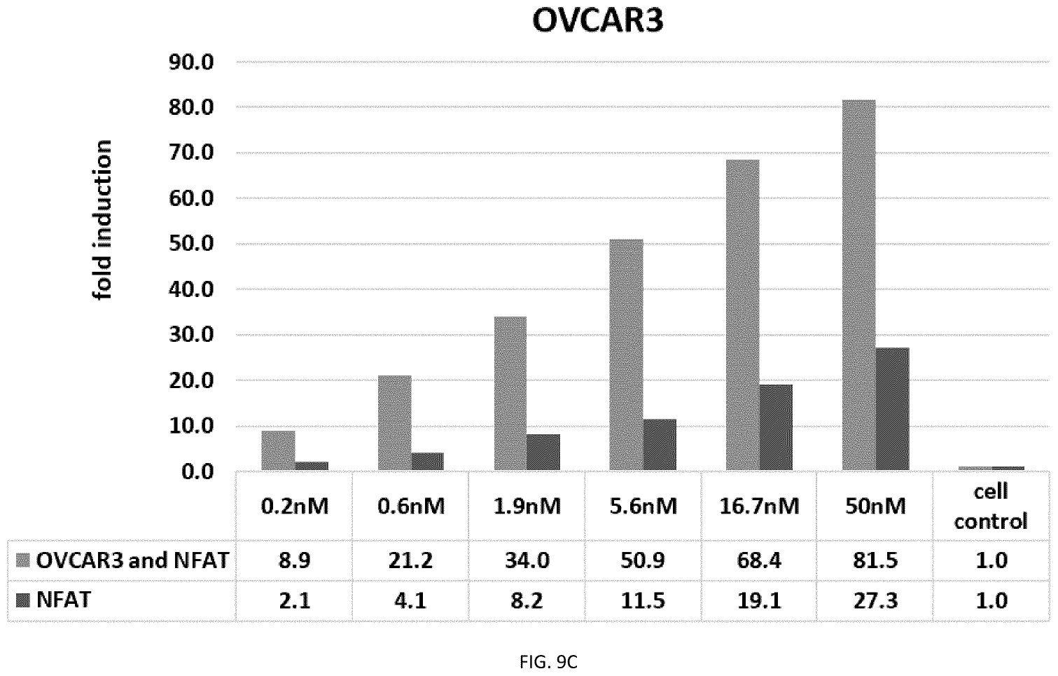

NFAT reporter (Luc)-Jurkat cells response to G2D03 BsAb in the presence of OVCAR3 target cells. G2D03 BsAb simultaneously binds CD3 expressed on T cells and GRP78 expressed on OVCAR3 target cells. With increasing concentrations of G2D03 BsAb, in the presence of OVCAR3 cells caused higher fold of luciferase activity (8.9-fold to 81.5-fold) than in the absence of OVCAR3 cells (2.1-fold to 27.3-fold) by activation of NFAT through CD3 signaling pathway. A schematic view is shown in FIG. 9A, and the results are shown in FIGS. 9B and 9C.

### 7-3 T Cell-Mediated Cytotoxicity

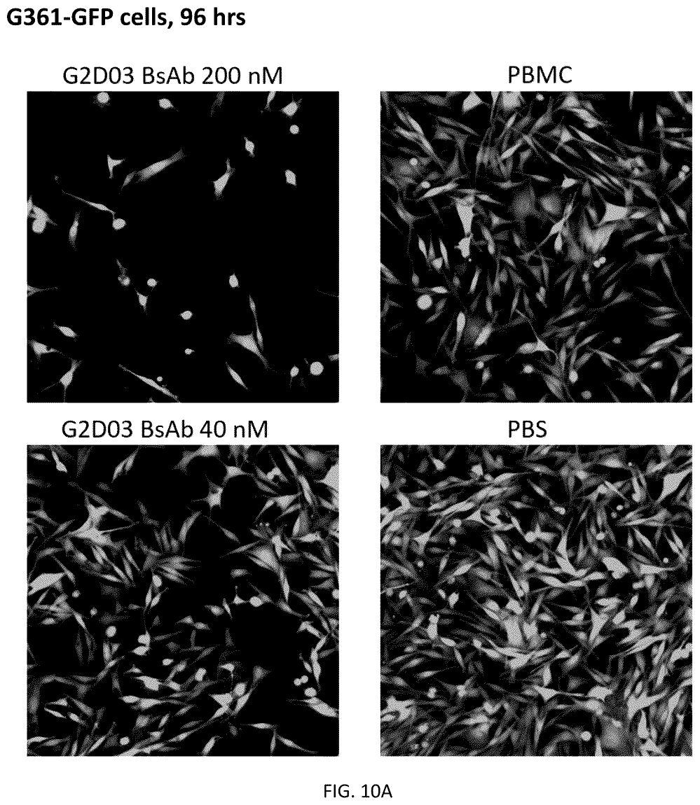

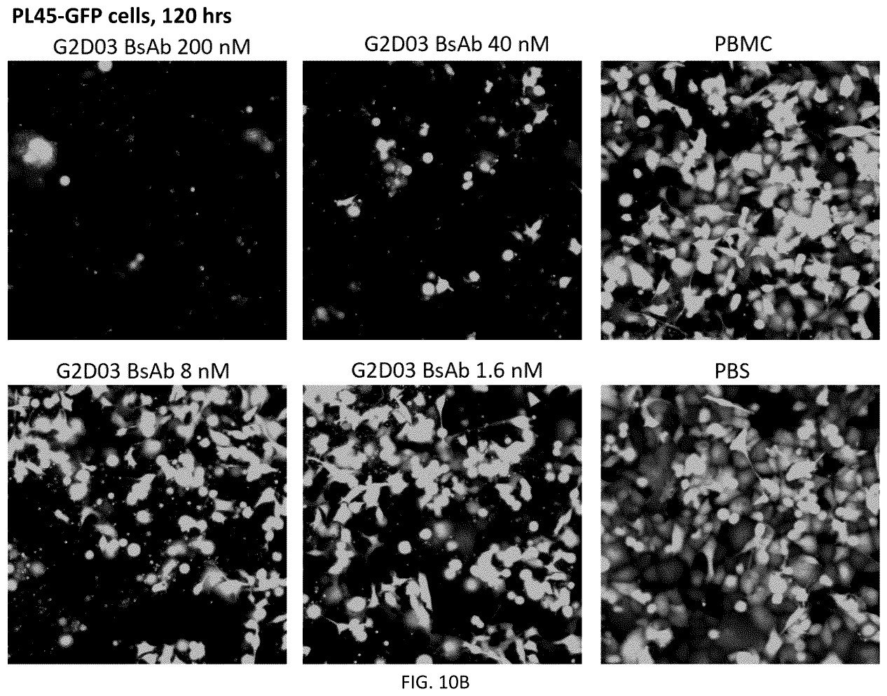

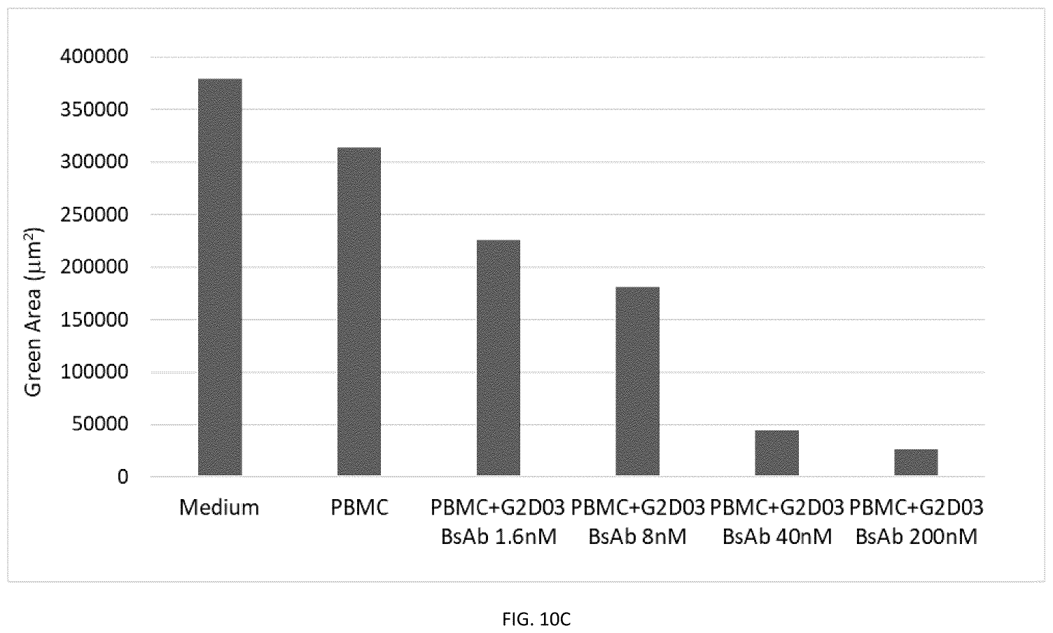

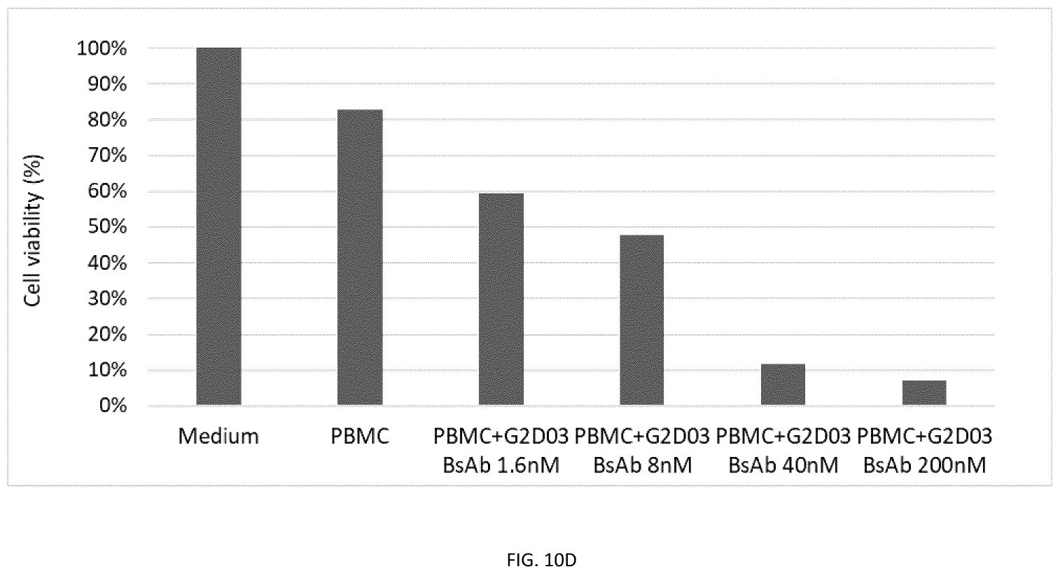

The effector cells, PBMC, were incubated with BsAbs on human cancer cell line G361-GFP at 40:1 E/T ratio for 96 and 120 hrs. PBS (medium) was used as negative control for BsAb. Cell viability was measured the GFP area (mm²) of cells and analyzed with Developer Toolbox 1.9.2 by IN Cell Analyzer 6000 (GE). The results are shown in FIGS. 10A to 10D.

8 Internalization of Anti-GRP78 mAbs in Different Type of Cancer Cells

Receptor-mediated internalization of antibodies can provide cell-specific drug delivery. The internalization is necessary for some targeted therapies using ADC. It is known in the art that internalization of ADCs is both antibody-dependent and payload-dependent. That is, not all antibodies can provide delivery mechanism for ADCs. Similarly, different payloads on the same antibody may have dramatically different internalization efficiencies.

Internalization and degradation of anti-GRP78 ADC and anti-GRP78 antibody can be measured by flow cytometry. Two methods were used in this study, directly fluorescent-labeled on antibodies or use fluorescent-conjugated secondary antibodies to detect the primary antibodies left on the cell surface after internalization.

Briefly, cancer cells were seeded in 1×10⁵ cell/well. Next, 0.5-1 mg of fluorescent-labelled anti-GRP78 antibody was subjected in 100 μl of target cell (cell density 1×10⁶ cells/ml) in FACS buffer for 1 hour at 4° C. to enable specific binding of anti-GRP78 antibody to the cell surface targets. After incubation, the cells were washed three times with FACS buffer to removed unbound antibody. The cells were then incubated at 37° C. with 5% CO₂ for antibody internalization.

Due to poor binding ability of C38 (EBIOSCIENCE™, Cat. No. 14-9768-82), it is hard to demonstrate its internalization. However, in some embodiments of this invention, G2D03 was internalized into ovarian cancer cells (OVCAR3), melanoma cells (G-361), and myeloma cells (RPMI 8226) during 37° C. incubation confirmed by flow cytometry. G2D03 (30 μg/mL) incubated at 37° C. to allow the antibodies to internalize, while incubated at 4° C. to prevent the internalization of the antibody. The samples were collected at various time points (30, 60, 90 and 120 mins) and determined by comparing the decrease cell surface-bound antibody of samples incubated at 37° C. to control samples incubated at 4° C.

1. An isolated antibody or antigen-binding fragment thereof that is specific for an epitope in glucose-regulated protein 78 (GRP78); wherein the isolated antibody or antigen-binding fragment thereof comprises complementarity determining regions (CDRs) of a heavy chain variable region or CDRs of a light chain variable region,
wherein the CDRs of the heavy chain variable region comprise:
CDRH1 having the amino acid sequence of SEQ ID NO: 1, CDRH2 having the amino acid sequence of SEQ ID NO: 2, and CDRH3 having the amino acid sequence of SEQ ID NO: 3; and
wherein the CDRs of the light chain variable region comprise:
CDRL1 having the amino acid sequence of SEQ ID NO: 4, CDRL2 having the amino acid sequence of SEQ ID NO: 5, and CDRL3 having the amino acid sequence of SEQ ID NO: 6.

2. The isolated antibody or antigen-binding fragment thereof of claim 1, wherein the heavy chain variable region comprises the amino acid sequence of SEQ ID NO: 7 or the amino acid sequence with at least about 95% identity to SEQ ID NO: 7, and the light chain variable region comprises the amino acid sequence of SEQ ID NO: 8 or the amino acid sequence with at least about 95% identity to SEQ ID NO: 8.

3. The isolated antibody or antigen-binding fragment thereof of claim 1, which is an Fab fragment, an F(ab′)₂ fragment, an ScFv fragment, a monoclonal antibody, a chimeric antibody, a humanized antibody or a human antibody.

4. The isolated antibody or antigen-binding fragment thereof of claim 3, which is an ScFv fragment comprising the heavy chain variable region having the amino acid sequence of SEQ ID NO: 7 or the amino acid sequence with at least about 95% identity to SEQ ID NO: 7, the light chain variable region having the amino acid sequence of SEQ ID NO: 8 or the amino acid sequence with at least about 95% identity to SEQ ID NO: 8, and a linker linking the heaving chain variable region and the light chain variable region, wherein the linker comprises the amino acid sequence of SEQ ID NO: 21 or 12, or the amino acid sequence with at least about 95% identity to SEQ ID NO: 21 or 12.

5. The isolated antibody or antigen-binding fragment thereof of claim 1, which is linked to a second specific binding domain for a second target, wherein the second specific binding domain comprises a T-cell targeting domain.

6. The isolated antibody or antigen-binding fragment thereof of claim 5, wherein the T-cell targeting domain is specific for CD3.

7. The isolated antibody or antigen-binding fragment thereof of claim 5, wherein the T-cell targeting domain comprises CDRs of a heavy chain variable region and CDRs of a light chain variable region, wherein the heavy-chain variable region comprises HCDR1 having the amino acid sequence of SEQ ID NO: 13, HCDR2 having the amino acid sequence of SEQ ID NO: 14, and HCDR3 having the amino acid sequence of SEQ ID NO: 15; and the light-chain variable region comprises LCDR1 having the amino acid sequence of SEQ ID NO: 16, LCDR2 having the amino acid sequence of SEQ ID NO: 17, and LCDR3 having the amino acid sequence of SEQ ID NO: 18.

8. The isolated antibody or antigen-binding fragment thereof of claim 5, wherein the T-cell targeting domain comprises the amino acid sequence of SEQ ID NO: 19 or the amino acid sequence with at least about 95% identity to SEQ ID NO: 19.

9. The isolated antibody or antigen-binding fragment thereof of claim 5, which comprises a first heavy chain having the amino acid sequence of SEQ ID NO: 9 or the amino acid sequence with at least about 95% identity to SEQ ID NO: 9, a second heavy chain having the amino acid sequence of SEQ ID NO: 10 or the amino acid sequence with at least about 95% identity to SEQ ID NO: 10, and a light chain having the amino acid sequence of SEQ ID NO: 11 or the amino acid sequence with at least about 95% identity to SEQ ID NO: 11.

10. The isolated antibody or antigen-binding fragment thereof of claim 5, which comprises the amino acid sequence of SEQ ID NO: 22 or 20, or the amino acid sequence with at least about 95% identity to SEQ ID NO: 22 or 20.

11. The isolated antibody or antigen-binding fragment thereof of claim 1, which is expressed on a surface of a cell.

12. The isolated antibody or antigen-binding fragment thereof of claim 1, which is conjugated with a therapeutic agent.

13. A vector encoding the isolated antibody or antigen-binding fragment thereof of claim 1.

14. A genetically engineered cell expressing the isolated antibody or antigen-binding fragment thereof of claim 1.

15. The genetically engineered cell of claim 14, which is an immune cell or a stem cell.

16. The genetically engineered cell of claim 15, which is a T cell.

17. A method for treating, prophylactic treating and/or preventing a disease and/or disorder caused by or related to GRP78 activity or signaling in a subject in need thereof, comprising administering a pharmaceutical composition to the subject in need thereof, wherein the pharmaceutical composition comprises an effective amount of the isolated antibody or antigen-binding fragment thereof of claim 1 and optionally a pharmaceutically acceptable carrier.

18. The method of claim 17, wherein the disease is a cancer selected from the group consisting of lung cancer, breast cancer, prostate cancer, colorectal cancer, gastric cancer, pancreatic cancer, ovarian cancer, hepatocellular carcinoma, renal cell carcinoma, testicular cancer, melanoma, leukemia, and papillary thyroid cancer.

19. A method for detecting expression of GRP78 or a cancer, comprising contacting the sample with the isolated antibody or antigen-binding fragment thereof of claim 1.

20. A kit for detecting GRP78 or a cancer in a sample, comprising the isolated antibody or antigen-binding fragment thereof of any of claim 1.
